(19) United States
(12) Patent Application Publication
(10) Pub. No.: US 2023/0030220 A1
(43) Pub. Date: Feb. 2, 2023

(54) SEPARATION OF SIALYLATED OLIGOSACCHARIDES FROM FERMENTATION BROTH

(71) Applicant: Glycom A/S, Hørsholm (DK)

(72) Inventors: Nikolay KHANZHIN, Humlebæk (DK); Getachew S. MOLLA, Hørsholm (DK); Martin MATWIEJUK, Hamburg (DE); Pierre CHASSAGNE, Beaumont (FR)

(21) Appl. No.: 17/757,646

(22) PCT Filed: Dec. 18, 2020
(86) PCT No.: PCT/IB2020/062166

§ 371 Date: Jun. 17, 2022

(30) Foreign Application Priority Data
Dec. 19, 2019 (DK) PA 2019 01595

(51) Int. Cl. C12P 19/04; B01D 61/14; B01D 61/58; B01D 69/02; B01D 71/56; C12P 19/26

(52) U.S. Cl. CPC C12P 19/04; B01D 61/147; B01D 61/58; B01D 69/02; B01D 71/56; C12P 19/26; C12Y 204/99; B01D 61/027

(57) ABSTRACT

The present invention relates to the separation and isolation of sialylated human milk oligosaccharides (HMOs) from the reaction mixture in which they are produced.

### FIELD OF THE INVENTION

The present invention relates to the separation and isolation of sialylated human milk oligosaccharides (HMOs) from the reaction mixture in which they are produced.

### BACKGROUND OF THE INVENTION

During the past decades, the interest in the preparation and commercialisation of human milk oligosaccharides (HMOs) has been increasing steadily. The importance of HMOs is directly linked to their unique biological activities, therefore HMOs have become important potential products for nutrition and therapeutic uses. As a result, low cost ways of producing industrially HMOs have been sought.

To date, the structures of more than 140 HMOs have been determined, and considerably more are probably present in human milk (Urashima et al.: Milk oligosaccharides, Nova Biomedical Books, 2011; Chen Adv. Carbohydr. Chem. Biochem. 72, 113 (2015)). The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GlcNAc). Lactose and the N-acetyllactosaminylated or lacto-N-biosylated lactose derivatives may further be substituted with one or more fucose and/or sialic acid residue(s), or lactose may be substituted with an additional galactose, to give HMOs known so far.

Sialylated human milk oligosaccharides such as disialyllacto-N-tetraose, 3′-O-sialyl-3-O-fucosyllactose, 6′-O-sialyllactose, 3′-O-sialyllactose, 6′-O-sialylated-lacto-N-neotetraose and 3′-O-sialylated-lacto-N-tetraose, are among the major components of human milk. In these sialylated human milk oligosaccharides the sialic acid residue is always linked to the 3-O- and/or 6-O-position of a terminal D-galactose or to the 6-O-position of a non-terminal GlcNAc residue via α-glycosidic linkages. Sialylated HMOs are thought to have significant health benefits for the neonate, because of their roles in supporting resistance to pathogens, gut maturation, immune function and cognitive development (ten Bruggencate et al. Nutr. Rev. 72, 377 (2014)).

Efforts to develop processes for synthesizing HMOs, including sialylated HMOs, have increased significantly in the last ten years due to their roles in numerous human biological processes. In this regard, processes have been developed for producing them by microbial fermentations, enzymatic processes, chemical syntheses, or combinations of these technologies. With regard to productivity, fermentation processes, on a lab scale, to produce 3′-SL and 6′-SL have proved to be promising.

However, to isolate sialylated lactoses or sialylated oligosaccharides from a complex matrix such as a fermentation broth is a challenging task. Antoine et al. Angew. Chem. Int. Ed. 44, 1350 (2005) and US 2007/0020736 disclosed the production of 3′-SL and accompanying di- and trisialylated lactoses by a genetically modified E. coli; the broth containing approx. 0.8 mM 3′-SL was treated as follows: adsorption of the products from the centrifuged supernatant on charcoal/celite, washing away the water soluble salts with distilled water, eluting the compounds by gradient aqueous ethanol, separation of the sialylated products on a Biogel column and desalting, leading to 49 mg of 3′-SL from 1 litre of broth. WO 01/04341 and Priem et al. Glycobiology 12, 235 (2002) disclosed the production of 3′-SL by a genetically modified E. coli; 3′-SL was isolated by the following sequence of operations: heat permeabilization of the producing cells followed by centrifugation, adsorption of the product from the supernatant on charcoal/celite, washing away the water soluble salts with distilled water, eluting the compound by gradient aqueous ethanol, binding the compound to a strong anion exchanger in HCO₃⁻-form, elution of the compound with a linear gradient NaHCO₃-solution, then eliminating the sodium bicarbonate with a cation exchanger (in H⁺-form), resulting in isolated 3′-SL with 49% purification yield. WO 2007/101862 and Fierfort et al. J. Biotechnol. 134, 261 (2008) disclosed an alternative work-up procedure of a 3′-SL fermentation broth, the procedure comprising the steps of heat permeabilization of the producing cell, centrifugation, adjusting the pH of the extracellular to 3.0 by the addition of a strong cation exchanger resin in acid form, removal of the precipitated proteins by centrifugation, adjusting the pH of the supernatant to 6.0 by the addition of a weak anion exchanger in base form, binding the sialyllactose to an anion exchanger in HCO₃⁻-form, after washing with distilled water, elution of the compound with a continuous gradient NaHCO₃-solution, eliminating the sodium bicarbonate with a cation exchanger (in H⁺-form) until pH 3.0 was reached, then adjustment of the pH to 6.0 with NaOH. The above purification allowed to isolate 15 g of 3′-SL from 1 litre of broth containing 25.5 g of 3′-SL. Drouillard et al. Carbohydr. Res. 345, 1394 (2010)) applied Fierfort's procedure above to a fermentation broth containing 6′-SL (11 g/l) and some 6,6′-disialyllactose (DSL), and thus isolated 3.34 g 6′-SL+DSL in a ratio of 155/86.

WO 2006/034225 describes two alternative purifications of 3′-SL from a producing fermentation broth. According to the first procedure, the lysate from the culture was diluted with distilled water and stirred with activated charcoal/celite. The slurry was washed with water, then the product was eluted from the charcoal/celite with aq. ethanol. According to the second method, the culture cells were heat treated and the precipitated solids were separated from the supernatant by centrifugation. The resulting supernatant was processed through a microfilter, the permeate was passed through a 10 kDa membrane, then nanofiltered. The resulting retentate was then diafiltered to collect the final sample. Both purification methods provided 90-100 mg 3′-SL from 1 litre of fermentation broth.

Both Gilbert et al. Nature Biotechnol. 16, 769 (1998) and WO 99/31224 disclose the enzymatic production of 3′-SL starting from lactose, sialic acid, phosphoenolpyruvate, ATP and CMP using a CMP-Neu5Ac synthetase/α-2,3-sialyl transferase fusion protein extract. The product was purified by a sequence of ultrafiltration (3000 MWCO), C18 reverse phase chromatography, nanofiltration/diafiltration at pH=3 and pH=7, acidification with a strong cation exchange (H+) resin, neutralization with NaOH solution and active charcoal decolourization.

WO 2009/113861 discloses a process for isolating sialyllactose from defatted and protein-free milk stream, comprising contacting said milk stream with a first anion exchange resin in the free base form and having a moisture content of 30-48% so that the negatively charged minerals are bound to the resin and the sialyllactose is not, followed by a treatment with a second anion exchange resin in the free base form which is a macroporous or gel type resin and has a moisture content between 50 and 70% so that the sialyllactose is bound to the resin. In this process, the sialyllactose containing stream is rather diluted (a couple of hundreds ppm of concentration) and the sialyllactose recovery from the first resin is moderate.

WO 2017/152918 discloses a method obtaining a sialylated oligosaccharide from a fermentation broth, wherein said sialylated oligosaccharide is produced by culturing a genetically modified microorganism capable of producing said sialylated oligosaccharide from an internalized carbohydrate precursor, comprising the steps of:

i) ultrafiltration (UF),

ii) nanofiltration (NF),

iii) optional activated charcoal treatment, and

iv) treating the broth with a strong anion exchange resin and/or cation exchange resin.

EP-A-3456836 discloses a method for separating a sialylated oligosaccharide from an aqueous medium, the method comprising a treatment of an aqueous solution containing said sialylated oligosaccharide with at least two types of an ion exchange resin, one being a strong anion exchange resin in Cl⁻-form and the other being a strong cation exchange resin.

WO 2019/043029 discloses a method for purifying sialylated oligosaccharides that have been produced by microbial fermentation or in vitro biocatalysis, the method comprising the steps of

i) separating biomass from the fermentation broth,

ii) removing cations from the fermentation broth or reaction mixture,

iii) removing anionic impurities from the fermentation broth or reaction mixture, and

iv) removing compounds having a molecular weight lower than that of the sialylated oligosaccharide to be purified from the fermentation broth or reaction mixture.

WO 2019/229118 discloses a method for the purification of a sialyllactose from other carbohydrates, the sialyllactose being produced by fermentation, comprising:

a) separating the cell-mass with ultrafiltration,

b) strong cationic ion exchanger treatment followed by strong anionic ion exchanger (Cl⁻-form) treatment of the filtrate,

c) first nanofiltration,

d) second nanofiltration,

e) electrodialysis,

f) reverse osmosis,

g) active charcoal treatment,

h) sterile filtration, and

i) spray-drying.

However, alternative procedures for isolating and purifying a sialylated HMO from non-carbohydrate components of the fermentation broth in which they have been produced, especially those suitable for industrial scale, are needed to improve the recovery yield of the HMO and/or to simplify prior art methods while the purity of the HMO is at least maintained, preferably improved.

### SUMMARY OF THE INVENTION

The invention relates to a method for obtaining or isolating or purifying a sialylated human milk oligosaccharide (HMO), preferably 3′-SL or 6′-SL, from the reaction milieu in which they have been produced, preferably from a fermentation broth, wherein said HMO has been produced by culturing a genetically modified microorganism capable of producing said HMO from an internalized carbohydrate precursor, comprising the steps of:
pre-treating the reaction milieu via pH-adjustment, dilution and/or heat treatment, and
centrifuging the reaction milieu after the pre-treatment or contacting the reaction milieu after the pre-treatment to an ultrafiltration (UF) membrane with a molecular weight cut-off (MWCO) of around 5-1000 kDa, wherein the membrane is preferably composed of a non-polymeric material.

In one embodiment, the method comprises
setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C., and
contacting the reaction milieu obtained in the previous step to an ultrafiltration (UF) membrane with a molecular weight cut-off (MWCO) of around 5-1000 kDa, such as 10-1000 kDa, wherein the membrane is preferably composed of a non-polymeric material.

In one embodiment, the method comprises
a) optionally, centrifugation or microfiltration of the reaction milieu, or filtration of the reaction milieu on a filter press or a drum filter,
b) as pre-treatment, setting the pH of the filtrate or supernatant from step a) or the reaction milieu directly to 3-6 and/or warming up the filtrate or supernatant from step a) or the reaction milieu directly to 35-65° C., and
c) contacting the reaction milieu obtained in step b) to an ultrafiltration (UF) membrane with a molecular weight cut-off (MWCO) of 5-1000 kDa, such as 10-1000 kDa, and collecting the permeate,

with the proviso that when step a) is not carried out, then the UF membrane is a non-polymeric membrane.

In one embodiment, step a) is not carried out and the non-polymeric membrane is a ceramic membrane.

In one embodiment, the method disclosed above further comprises a nanofiltration step.

In other embodiment, the method disclosed above further comprises a treatment with one or more ion exchange resins.

In other embodiment, the method disclosed above further comprises a treatment with or chromatography on active charcoal.

One embodiment of the method relates to obtaining, isolating or purifying a sialylated HMO, preferably 3′-SL or 6′-SL, from the reaction milieu in which it has been produced, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, such as 10-1000 kDa, wherein the membrane is preferably composed of a non-polymeric material, and
iii) contacting the permeate obtained in step ii) with a nanofiltration membrane.

One embodiment of the method relates to obtaining, isolating or purifying a sialylated HMO, preferably 3′-SL or 6′-SL, from the reaction milieu in which it has been produced, comprising the steps of:
i) pre-treating the reaction milieu via pH-adjustment, dilution and/or heat treatment,
ii) centrifuging the reaction milieu after the pre-treatment or contacting the reaction milieu after the pre-treatment to an ultrafiltration (UF) membrane with a molecular weight cut-off (MWCO) of around 5-1000 kDa, such as 10-1000 kDa, wherein the membrane is preferably composed of a non-polymeric material, and
iii) contacting the clarified supernatant or the UF permeate obtained in step ii) with a nanofiltration membrane.

One embodiment of the method relates to obtaining, isolating or purifying a sialylated HMO, preferably 3′-SL or 6′-SL, from the reaction milieu in which it has been produced, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, such as 10-1000 kDa, wherein the membrane is preferably composed of a non-polymeric material,
iii) contacting the permeate obtained in step ii) with a nanofiltration (NF) membrane, and
iv) treating the NF retentate obtained in step iii) with an ion exchange resin, for example a strong cationic ion exchange resin and a weak basic ion exchange resin, to demineralize the retentate.

One embodiment of the method relates to obtaining, isolating or purifying a sialylated HMO, preferably 3′-SL or 6′-SL, from the reaction milieu in which it has been produced, comprising the steps of:
i) pre-treating the reaction milieu via pH-adjustment, dilution and/or heat treatment,
ii) centrifuging the reaction milieu after the pre-treatment or contacting the reaction milieu after the pre-treatment to an ultrafiltration (UF) membrane with a molecular weight cut-off (MWCO) of around 5-1000 kDa, such as 10-1000 kDa, wherein the membrane is preferably composed of a non-polymeric material,
iii) contacting the clarified supernatant or the UF permeate obtained in step ii) with a nanofiltration membrane, and
iv) treating the NF retentate obtained in step iii) with an ion exchange resin, for example a strong cationic ion exchange resin and a weak basic ion exchange resin, to demineralize the retentate.

One embodiment of the method relates to obtaining, isolating or purifying a sialylated HMO, preferably 3′-SL or 6′-SL, from the reaction milieu in which it has been produced, comprising the steps of:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-85° C.,
ii) contacting the reaction milieu obtained in step i) to an ultrafiltration (UF) membrane with a molecular weight cut-off (MWCO) of around 5-1000 kDa, such as 10-1000 kDa, wherein the membrane is preferably composed of a non-polymeric material,
iii) contacting the permeate obtained in step ii) with a nanofiltration (NF) membrane,
iv) treating the retentate obtained in step iii) with an ion exchange resin, for example a strong cationic ion exchange resin and a weak basic ion exchange resin, to demineralize the retentate, and
v) the solution obtained in step iv) is contacted with activated charcoal (AC).

One embodiment of the method relates to obtaining, isolating or purifying a sialylated HMO, preferably 3′-SL or 6′-SL, from the reaction milieu in which it has been produced, comprising the steps of:
i) pre-treating the reaction milieu via pH-adjustment, dilution and/or heat treatment,
ii) centrifuging the reaction milieu after the pre-treatment or contacting the reaction milieu after the pre-treatment to an ultrafiltration (UF) membrane with a molecular weight cut-off (MWCO) of around 5-1000 kDa, such as 10-1000 kDa, wherein the membrane is preferably composed of a non-polymeric material,
iii) contacting the clarified supernatant or the UF permeate obtained in step ii) with a nanofiltration membrane,
iv) treating the NF retentate obtained in step iii) with an ion exchange resin, for example a strong cationic ion exchange resin and a weak basic ion exchange resin, to demineralize the retentate, and
v) the solution obtained in step iv) is contacted with activated charcoal (AC).

Preferably, the reaction milieu in which the sialylated HMO, preferably 3′-SL or 6′-SL, has been produced is a fermentation broth. The fermentation broth typically contains, besides the sialylated HMO of interest as main compound for the production of which a genetically modified microorganism, preferably an E. coli, has been suitably designed, carbohydrate by-products or contaminants such as carbohydrate intermediates in the biosynthetic pathway of the sialylated HMO of interest from lactose, preferably exogenously added lactose, as precursor, and/or those as a result of a deficient, defective or impaired glycosylation during the biosynthetic pathway, and/or those as a result of rearrangement or degradation under the cultivation condition or post-fermentative operations, and/or lactose as unconsumed educt added in excess during the fermentation. Further, the fermentation broth can contain cells, proteins, protein fragments, DNA, caramelized by-products, minerals, salts, organic acids, endotoxins and/or other charged molecules.

Preferably, when the fermentation broth, before UF, is not subjected to centrifugation, microfiltration or filtration on a filter press or a drum filter, the UF membrane is composed of a non-polymeric material (e.g. the UF membrane is a ceramic membrane).

Certain embodiments of the invention comprise one or more further optional steps. Preferably, the further optional step is not electrodialysis.

In one embodiment, preferably when the UF permeate is poor in or essentially lacks lactose, the NF step according to step iii) comprises the use of a nanofiltration membrane that has a MWCO that ensures the retention of the sialylated human milk oligosaccharide of interest, that is its MWCO is around 25-50% of the molecular weight of the sialylated human milk oligosaccharide, typically around 150-500 Da. In this regard the sialylated human milk oligosaccharide is accumulated in the NF retentate (NFR), whereas salts such as monovalent ions or monosaccharides are accumulated in the permeate.

In other embodiment, preferably when the UF permeate contains substantial amount of lactose, the NF step according to step iii) comprises the use of a nanofiltration membrane with a MWCO of around 600-3500 Da, preferably around 1000 Da, ensuring the retention of the sialylated HMO and allowing mono- and divalent salts and at least a part of the lactose to pass through the membrane, wherein the active (top) layer of the NF membrane is composed of polyamide, and wherein the MgSO₄ rejection factor on the NF membrane is around 50-90%.

The invention relates, in another aspect, to the separation of a sialylated HMO from dissolved inorganic and organic salts, acids and bases in an aqueous medium from a fermentation or enzymatic process, comprising the step of subjecting the aqueous medium to steps i), ii), iii), iv) and optionally step v).

The invention relates, in a third aspect, to separating a sialylated HMO, preferably 3′-SL or 6′-SL, from an aqueous medium, the method comprising a treatment of the aqueous solution containing said sialylated HMO with at least two types of an ion exchange resin, one being a weak anion exchange resin in base form and the other being a strong cation exchange resin in H⁺-form.

Preferably, said aqueous medium is a fermentation broth or an enzymatic reaction mixture containing said sialylated HMO.

Preferably, the separated sialylated HMO is obtained in the form of its alkali metal salt, preferably sodium salt.

Preferably, the weak anion exchange resin treatment follows, preferably directly follows, the strong cation exchange resin treatment.

In one embodiment of the third aspect of the invention, the ion exchange treatments are preceded by pre-treatment steps of the aqueous medium or aqueous solution containing the sialylated oligosaccharide, wherein the pre-treatment steps comprise ultrafiltration, nanofiltration and optional active charcoal treatment, preferably in the following order: ultrafiltration, nanofiltration and optional active charcoal treatment.

### DETAILED DESCRIPTION OF THE INVENTION

### 1. Terms and Definitions

The term “sialylated human milk oligosaccharide” means a sialylated complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that can be elongated by one or more β-N-acetyl-lactosaminyl and/or one or more β-lacto-N-biosyl units, and which core structure is substituted by an α-N-acetyl-neuraminyl (sialyl) moiety and optionally can be substituted by an a L-fucopyranosyl moiety. In this regard, the acidic HMOs have at least one sialyl residue in their structure. Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DS-LNT).

The term “genetically modified cell” or “genetically modified microorganism” preferably means a cell of a microorganism, such as a bacterial or fungi cell, e.g. an E. coli cell, which has been genetically manipulated to include at least one alteration in its DNA sequence. The term “at least one genetic alteration” means a genetic alteration that can result in a change in the original characteristics of the wild type cell, e.g. the modified cell is able to perform additional chemical transformation due to the introduced new genetic material that encodes the expression of an enzymes not being in the wild type cell, or is not able to carry out transformation like degradation due to removal of gene/genes (knockout). A genetically modified cell can be produced in a conventional manner by genetic engineering techniques that are well-known to those skilled in the art.

The term “genetically modified cell or microorganism capable of producing a sialylated oligosaccharide from an internalized carbohydrate precursor” preferably means a cell or a microorganism which is genetically manipulated (vide supra) to comprise a recombinant gene encoding a sialyl transferase necessary for the synthesis of said sialylated oligosaccharide, a biosynthetic pathway to produce a sialic acid nucleotide donor suitable to be transferred by said glycosyl transferase to a carbohydrate precursor (acceptor) and/or a mechanism of internalization of a carbohydrate precursor (acceptor) from the culture medium into the cell where it is sialylated to produce the sialylated oligosaccharide of interest.

The term “biomass”, in the context of fermentation, refers to the suspended, precipitated or insoluble materials originating from fermentation cells, like intact cells, disrupted cells, cell fragments, proteins, protein fragments, polysaccharides. The term “biomass”, in the context of enzymatic reaction, refers to (mainly denatured and/or precipitated) proteins or protein fragments originating from the enzyme used. The biomass can be separated from the above reaction mixture (suspension) by e.g. centrifugation, microfiltration, ultrafiltration or filtration on a filter press or a drum filter.

The term “Brix” refers to degrees Brix, that is the sugar content of an aqueous solution (g of sugar in 100 g of solution). In this regard, Brix of the sialylated oligosaccharide solution of this application refers to the overall carbohydrate content of the solution including the sialylated oligosaccharide and its accompanying carbohydrates. Brix is measured by a calibrated refractometer.

Rejection factor of a salt (in percent) is calculated as (1−κₚ/κᵣ) 100, wherein κₚ is the conductivity of the salt in the permeate and κᵣ is the conductivity of the salt in the retentate. The retentate concentration is practically equal to the feed concentration concerning the salt. The procedure for measuring rejection of salts is disclosed in the working examples below.

Rejection factor of a carbohydrate (in percent) is calculated as (1−Cₚ/Cᵣ) 100, wherein Cₚ is the concentration of the carbohydrate in the permeate and Cᵣ is the concentration of the carbohydrate in the retentate. The retentate concentration is practically equal to the feed concentration concerning the carbohydrate. One exemplary procedure for measuring rejection of a carbohydrate is disclosed in the working examples below.

Separation factor concerning two carbohydrates is calculated as (Cₚ₁/Cᵣ₁)/(Cₚ₂/Cᵣ₂), wherein Cₚ₁ and Cₚ₂ are the concentrations of the first and the second carbohydrate, respectively, in the permeate, and Cᵣ₁ and Cᵣ₂ are the concentrations of the first and the second carbohydrate, respectively, in the retentate.

“Pure water flux” is defined as the volume of purified water (e.g. distilled water, RO water) that passes through a membrane per unit time, per unit area and per unit of transmembrane pressure under specified conditions (at around 23-25° C., 10 bar and constant cross-flow of 300 l/h).

“Demineralization” preferably means a process of removing minerals or mineral salts from a liquid. In the context of the present invention, demineralization preferably refers to the step of ion exchange treatment, especially a subsequent application of a cation and an anion exchange resin so that the eluate from the second ion exchanger contains no or very low amount of minerals or minerals salts. Moreover, demineralization can occur in the nanofiltration step, especially when it is combined with diafiltration.

“Microfiltration” preferably means a pre-treatment separation processes to filter a fermentation broth or an enzymatic reaction mixture through a membrane having pore ranging from about 0.1 to 10 m. In terms of approximate molecular weight, these membranes can separate macromolecules of molecular weights generally less than 100,000 g/mol.

The terms “around” or “about” used throughout the specification of the invention in connection with a numerical value mean that said numerical value may deviate up to 10% of the indicated value.

### 2. Method for Obtaining or Isolating a Sialylated HMO from the Reaction Milieu in which it has been Produced

The invention relates to a method for obtaining, isolating or purifying a sialylated HMO from an aqueous medium, the aqueous medium being a fermentation broth or an enzymatic reaction mixture in which said sialylated HMO has been produced. The reaction milieu is a complex matrix in which the sialylated HMO is accompanied or contaminated by several substances like by-product and residual materials necessary for the synthesis of said sialylated HMOs. Accordingly, the sialylated HMO is obtained, isolated or purified from the reaction milieu by separating it from the by-products and residual materials with the aid of several consecutive steps resulting in that the sialylated HMO is obtained or isolated in much purer form than it was in the reaction milieu. The present invention thus provides a purification method by which the sialylated HMO of interest can be obtained or isolated in a more beneficial way compared to the prior art. Such benefits are disclosed below with respect to the corresponding method steps.

Using a biotechnological method, whatever the way (fermentation or in vitro enzymatic) how the sialylated HMO is produced, the reaction milieu contains a biomass. Therefore, the method of invention compulsorily comprises a step of separating the biomass from the reaction milieu to provide an aqueous solution comprising the sialylated HMO of interest. Separating the biomass from the reaction milieu comprises ultrafiltration (UF, see details below) optionally preceded by pre-filtration (that is centrifugation, microfiltration, or filtration on a filter press or a drum filter) or centrifuging. The UF or centrifuging is usually followed by a nanofiltration step (NF, see details below). Further, the method of invention can optionally comprise a treatment with ion exchange resins, advantageously with a cation and an anion exchange resin. In addition, the method of invention can optionally comprise active charcoal treatment for decolorizing. Any of the optional steps can be performed at any order after UF and NF. Furthermore, the method of invention can optionally comprise at least one more NF step, especially for concentrating and or desalinating/demineralizing the aqueous solution of the sialylated HMO. Alternatively, if demineralization is not necessary (e.g. due to low salt content of the feed), the optional additional NF may be replaced by evaporation.

The steps of UF, NF, “treatment with ion exchange resins” and “active charcoal treatment” are discussed in detail below in the corresponding sub-chapters.

Accordingly, the method comprises the following separation/purification steps in any order:

aa) centrifuging (CF) or ultrafiltration (UF),

bb) nanofiltration (NF), and

cc) treatment with an ion exchange resin.

Advantageously, step aa) is conducted before step bb). More advantageously, the step aa) is conducted before any of the steps bb) and cc). Preferably, the method is performed in the order where step bb) follows step aa) and step cc) follows step bb).

In one embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR), and
treatment of the NFR with ion exchange resins and collecting the resin eluate (RE).

In one embodiment, the method comprises:
centrifuging (CF) the reaction milieu and collecting the clarified supernatant,
nanofiltration (NF) of the clarified supernatant and collecting the nanofiltration retentate (NFR), and
treatment of the NFR with ion exchange resins and collecting the resin eluate (RE).

The method of the invention may comprise an active charcoal treatment after UF, NF or ion exchange resin treatment.

In one embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
treatment of the CCE with ion exchange resins.

In one embodiment, the method comprises:
centrifuging (CF) the reaction milieu and collecting the clarified supernatant,
nanofiltration (NF) of the clarified supernatant and collecting the nanofiltration retentate (NFR),
activated charcoal treatment of the NFR and collecting the charcoal eluate (CCE), and
treatment of the CCE with ion exchange resins.

Preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
activated charcoal treatment of the NFR and collecting the charcoal eluate (CCE), and
treatment of the CCE with a strong cation exchange resin in H⁺-form and a weak anion exchange resin in free base form.

Preferably, the method comprises:
centrifuging (CF) the reaction milieu and collecting the clarified supernatant,
nanofiltration (NF) of the clarified supernatant and collecting the nanofiltration retentate (NFR),
activated charcoal treatment of the NFR and collecting the charcoal eluate (CCE), and
treatment of the CCE with a strong cation exchange resin in H⁺-form and a weak anion exchange resin in free base form.

In another embodiment, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with ion exchange resins and collecting the resin eluate (RE), and
activated charcoal treatment of the RE.

In another embodiment, the method comprises:
centrifuging (CF) the reaction milieu and collecting the clarified supernatant,
nanofiltration (NF) of the clarified supernatant and collecting the nanofiltration retentate (NFR),
treatment of the NFR with ion exchange resins and collecting the resin eluate (RE), and
activated charcoal treatment of the RE.

Preferably, the method comprises:
ultrafiltration (UF) of the reaction milieu or the centrifuged reaction milieu and collecting the ultrafiltration permeate (UFP),
nanofiltration (NF) of the UFP and collecting the nanofiltration retentate (NFR),
treatment of the NFR with a strong cation exchange resin in H⁺-form and a weak anion exchange resin in free base form, and collecting the resin eluate (RE), and
activated charcoal treatment of the RE.

Preferably, the method comprises:
centrifuging (CF) the reaction milieu and collecting the clarified supernatant,
nanofiltration (NF) of the clarified supernatant and collecting the nanofiltration retentate (NFR),
treatment of the NFR with a strong cation exchange resin in H⁺-form and a weak anion exchange resin in free base form, and collecting the resin eluate (RE), and
activated charcoal treatment of the RE.

In one preferred embodiment, the method comprises the following sequence of steps consisting of steps A), B), C) and D):
A)—setting the pH of the reaction milieu, which is a fermentation broth, to a pH of about 3 to 5,
B)—centrifuging (CF) the pH-adjusted broth of step A) to give a clarified supernatant, or
   ultrafiltration (UF) of the pH-adjusted broth of step A) to give an ultrafiltration permeate (UFP),
C)—the clarified supernatant or UFP is treated with granulated active carbon to give a charcoal eluate (CCE), and
D)—nanofiltration (NF) of the CCE and collecting the nanofiltration retentate (NFR),

wherein the method does not comprise ion exchange treatment, simulated moving bed chromatography, gel filtration/chromatography and electrodialysis.

The method of the invention provides a solution highly enriched with the sialylated HMO of interest from which that HMO can be obtained in high yield and preferably with a satisfactory purity, such as that meets the strict regulatory requirements for food applications.

2.1. Production of the Sialylated HMO

2.1.1 Production of the Sialylated HMO by a Genetically Modified Microorganism

The production of the sialylated HMO by culturing a genetically modified cell is preferably performed as the following.

The fermentative production comprising a genetically modified cell preferably occurs in the following way. An exogenously added acceptor is internalized from the culture medium into the cell where it is converted to the sialyl oligosaccharide of interest in a reaction comprising enzymatic sialylation mediated by an appropriate sialyl transferase. In one embodiment, the internalization can take place via a passive transport mechanism during which the exogenous acceptor diffuses passively across the plasma membrane of the cell. The flow is directed by the concentration difference in the extra- and intracellular space with respect to the acceptor molecule to be internalized, which acceptor is supposed to pass from the place of higher concentration to the zone of lower concentration tending towards equilibrium. In another embodiment, the exogenous acceptor can be internalized in the cell with the aid of an active transport mechanism, during which the exogenous acceptor diffuses across the plasma membrane of the cell under the influence of a transporter protein or permease of the cell. Lactose permease (LacY) has specificity towards mono- or disaccharide selected from galactose, N-acetyl-glucosamine, a galactosylated monosaccharide (such as lactose), an N-acetyl-glucosaminylated monosaccharide and glycosidic derivatives thereof. All these carbohydrate derivatives can be easily taken up by a cell having a LacY permease by means of an active transport and accumulate in the cell before being glycosylated (WO 01/04341, Fort et al. J. Chem. Soc., Chem. Comm. 2558 (2005), EP-A-1911850, WO 2013/182206, WO 2014/048439). This is because the cell is able to transport these carbohydrate acceptors into the cell using its LacY permease, and the cell lacks any enzymes that could degrade these acceptors, especially LacZ. The specificity towards the sugar moiety of the substrate to be internalized can be altered by mutation by means of known recombinant DNA techniques. In a preferred embodiment, the exogenously added acceptor is lactose, and its internalization takes place via an active transport mechanism mediated by a lactose permease of the cell, more preferably LacY. Being internalized in the cell, the acceptor is sialylated by means of a sialyl transferase expressed by a heterologous gene or nucleic acid sequence which is introduced into the cell by known techniques, e.g. by integrating it into the chromosome of the cell or using an expression vector. The genetically modified cell comprises a biosynthetic pathway to produce a sialic acid monosaccharide nucleotide donor (typically CMP-sialic acid) suitable to be transferred by the corresponding sialyl transferase. The genetically modified cell can produce CMP-sialic acid, in two ways. In one way, exogenously added sialic acid is internalized actively or passively, preferably actively by a sialic acid permease, more preferably by that encoded by nanT, and subsequently converted to CMP-sialic acid by a CMP-NeuAc synthase, e.g. encoded by a heterologous neuA. In another way the internally available UDP-GlcNAc is utilized, by expressing heterologous neuC, neuB and neuA that convert it to CMP-sialic acid via ManNAc and sialic acid as intermediates. In the meantime, the cell's catabolic activity on sialic acid and its precursor is suppressed by inactivating/deletion of the aldolase gene (nanA) and/or the ManNAc kinase gene (nanK). The internalized carbohydrate precursor can be the subject of glycosylation other than sialylation, e.g. N-acetylglucosaminylation, galactosylation and/or fucosylation before being sialylated as described above.

In a preferred embodiment of the production of the sialylated HMO by a genetically modified microorganism, the microorganism able to produce the sialylated HMO is an E. coli, preferably of LacY⁺LacZ⁻ genotype carrying neuBCA. The heterologous sialyl transferase gene in the microorganism is preferably an α-2,3- or an α-2,6-sialyl transferase with the aid of which, e.g. from the exogenously added lactose as carbohydrate acceptor, 3′-SL or 6′-SL is produced, respectively. Such a microorganism is disclosed e.g. in WO 2007/101862, Fierfort et al. J. Biotechnol. 134, 261 (2008), Drouillard et al. Carbohydr. Res. 345, 1394 (2010) and WO 2017/101958.

In preferred embodiments, the genetically modified microorganism is E. coli.

Accordingly, in a preferred embodiment, the production process comprises the following steps:
providing a genetically modified E. coli cell of LacY⁺ phenotype or LacZ⁻, LacY⁺ phenotype, wherein said cell comprises:
   recombinant genes encoding a sialyl transferase necessary for the synthesis of the sialylated HMO, and
   one or more genes encoding a biosynthetic pathway to CMP-sialic acid for the sialyl transferase, and
culturing the genetically modified E. coli cell of LacY⁺ phenotype or LacZ⁻, LacY⁺ phenotype in the presence of exogenous lactose and a suitable carbon source, thereby producing a fermentation broth comprising the sialylated HMO.

The fermentation broth so-produced comprises the sialylated HMO both in the producing cells and the culture medium. To harvest the intracellular sialylated HMO and thereby to raise the titre of the product, the method described above may further comprise an optional step of disrupting or permeabilizing the cells, e.g. by heating.

The fermentation broth that comprises the sialylated HMO can be accompanied by other carbohydrate compounds. Typically, another carbohydrate compound is lactose which is used as acceptor in the fermentation process for making the sialylated HMO and left unconverted. In addition, another accompanying carbohydrate compound can be an intermediary carbohydrate during the biosynthetic pathway to the desired sialylated HMO, e.g. lacto-N-triose II, LNT or LNnT in case of making LSTs. Although their amounts can be substantially reduced in the fermentation broth before subjecting it to the separation/purification steps disclosed below, e.g. as disclosed in WO 2012/112777 or WO 2015/036138, it is not necessary to do so. The claimed method, in one embodiment, is suitable to separate a sialylated HMO accompanied by carbohydrate compounds from non-carbohydrate contaminants, while the relative proportion of the carbohydrate compounds does not substantially change in the course of the claimed method. Therefore the purpose of the claimed method, in one aspect, is a separation of a sialylated HMO accompanied by carbohydrate compounds from non-carbohydrate contaminants in an aqueous medium from fermentation broth or enzymatic reaction milieu rather than the purification of the sialylated HMO from any contaminants including accompanying carbohydrate compounds. Sialylated human milk oligosaccharides are intended to be used for nutritional purposes, therefore the presence of accompanying carbohydrates besides the main sialylated HMO in the final nutritional composition is not adverse, or it can even be advantageous. Another embodiment of the claimed method, however, is suitable to purify the sialylated HMO by separating it from carbohydrate and non-carbohydrate contaminations, thereby providing the sialylated HMO in substantially pure form.

Moreover, there can be further non-HMO carbohydrate contaminants in the fermentation broth. These are typically lactulose and its glycosylated derivatives. Lactulose may be formed from lactose by rearrangement when lactose is heat-sterilized before adding it to the fermentation and/or during the fermentation. As lactulose is also internalized by the cell, it can be glycosylated, similar to lactose, in a concurrent biotransformation reaction. However, the amount of lactulose and its glycosylated derivatives does not exceed a couple of tenth weight % of the overall dry solid matter of the broth after biomass separation.

According to the invention, the fermentation broth is further subjected to a procedure of separation/purification of the sialylated HMO from other non-carbohydrate compounds and optionally from carbohydrate compounds of the broth which is described below.

2.1.2 Production of the Sialylated HMO by Means of Ex Vivo Enzymatic Reaction

For ex vivo enzymatic synthesis of sialylated lactoses by using a transsialidase see e.g. Maru et al. Biosci. Biotech. Biochem. 56, 1557 (1992), Masuda et al. J. Biosci. Bioeng. 89, 119 (2000), WO 2012/007588, WO 2012/156897 or WO 2012/156898. For ex vivo enzymatic synthesis of 3′-SL by using a sialyl transferase see e.g. WO 96/32492, Gilbert et al. Nature Biotechnol. 16, 769 (1998), WO 99/31224 or Mine et al. J. Carbohydr. Chem. 29, 51 (2010).

According to the invention, the enzymatic reaction mixture is further subjected to a procedure of separation/purification of the sialylated HMO from other non-carbohydrate compounds and optionally from carbohydrate compounds which is described below.

2.2 Obtention of the Sialylated HMO from the Reaction Milieu in which it has been Produced

2.2.1. Optional Pre-Treatment of the Reaction Milieu Before Ultrafiltration

A fermentation broth typically contains, besides the sialylated HMO produced, the biomass of the cells of the used microorganism together with proteins, protein fragments, DNA, DNA fragments, endotoxins, biogenic amines, inorganic salts, unreacted carbohydrate acceptors such as lactose, sugar-like by-products, monosaccharides, colorizing bodies, etc. Optionally, in order to make macromolecules of the fermentation broth or the enzymatic reaction mixture more easily filterable, the reaction milieu is subjected to the a pH adjustment to around 2.5-7.5 and/or subjected to heat treatment between 30-75° C. and/or clarified by flocculation/coagulation. Also optionally, the reaction milieu, either treated as disclosed above or not, is centrifuged, microfiltered or filtered on a filter press or a drum filter, thereby at least a part of the biomass or the precipitated/flocculated/denatured enzyme(s) is/are removed.

The term “pre-treated reaction milieu” used in the context of the present invention comprises thus at least one of the above steps.

2.2.2 Pre-Treatment Before Centrifugation

The methods can include centrifugation to separate the biomass from the suspension. Further, the suspension may be pre-treated prior to any centrifugation. Advantageously, embodiments of the disclosure can increase sedimentation rate of the non-dissolved particles not by just few folds, but substantially by several orders of magnitude, e.g. 1000-10000-fold, compared to centrifuging without pre-treatment. Further, a clear supernatant (e.g. solution) containing the dissolved sialylated HMO can be produced.

In certain implementations, the suspension may be pre-treated by pH adjustment. In certain implementations, the suspension may be pre-treated by dilution. In certain implementations, the suspension may be pre-treated by heating. In certain variations, two or three of the disclosed pre-treatments can be performed. All pre-treatment disclosed below can be performed prior to centrifuging.

In particular, embodiments of the disclosure can be used for the separation of a liquid, preferably aqueous, phase containing a sialylated HMO from a suspension such as a fermentation broth.

Advantageously, the pre-treatment can be performed without adding a flocculation agent (e.g. an agent that accelerates the process such as the sedimentation rate such as inorganic polyvalent metal salts or charged polymers (e.g. polyethyleneimine (PEI) or chitosan)). Thus, no flocculation agent may be used in any or all embodiments of the disclosure.

Typical centrifuging of the solution without embodiments of the disclosed pre-treatments required long centrifugation (residence) time at high g-force in lab-scale trials with unspecified yields and unspecified efficacy of removal of proteins and other biomolecules and unspecified degree of clarification, such as by OD600 measurements. Thus, the known processes could not be used to effectively commercialize manufacturing, as only lab-scale operations could be performed.

However, through the use of embodiments of the disclosed pre-treatment methods, a number of advantages have been produced, such as 1) a substantially higher throughput is achieved; 2) a better yield of the dissolved sialylated HMO, is achieved due to dilution and heat treatment, both of which minimize the biomass/supernatant ratio and facilitate extraction of product from biomass; 3) fine particle removal after pre-treatment is much more efficient as quantified by OD600; 4) biomolecule removal is also more efficient (e.g. proteins due to denaturation and precipitation at lower pH and heat treatment); 5) higher throughput, i.e. higher flux could be achieved in the subsequent optional membrane filtration step such as microfiltration or ultrafiltration.

Further, embodiments of the disclosure have shorter step durations, smaller and less complex equipment, less cleaning chemicals, and require shorter and less frequent cleaning.

As will be discussed in further detail below, the pre-treatment of the suspension can include pH adjustment, and/or dilution, and/or heat treatment. In certain implementations, all three of pH adjustment, dilution, and heat treatment can be performed. In alternative embodiments, pH adjustment and dilution can be performed. In alternative embodiments, pH adjustment and heat treating can be performed. In alternative embodiments, heat treating and dilution can be performed.

Advantageously, a combination of a plurality of pre-treatment methods can provide an improved synergistic effect not found in individual pre-treatments.

All pre-treatment can be performed prior to centrifuging, or otherwise separating the sialylated HMOs, of the pre-treated suspension. In certain embodiments, one or more pre-treatment steps can occur during the centrifuging. For example, between steps in a multi-step centrifuging.

Alternatively, the centrifuging vessel can heat the suspension during centrifuging.

Advantageously, the pre-treatment can increase the settling velocity of the solid particles (biomass) in the suspension (broth) by 100-20000 fold making the biomass separation by centrifugation much more efficient and thus applicable in industrial scale. In addition to settling velocity, at least 3 other parameters are substantially improved due to pre-treatment:
1) the yield of the dissolved sialylated HMO in the liquid phase (supernatant) could be increased to >90% compared to only around 50-70% without pre-treatment after single centrifugation;
2) the protein and DNA content in supernatant could be reduced by around 10-fold;
3) the residual suspended solid content is also substantially reduced to OD600<0.1, such as OD600<0.05, e.g. to OD600 around 0.024, by centrifugation at low RCF=around 3000 g for 3 min compared to OD600>1 at around 10000 g/10 min for the untreated broth.

2.2.2.1 Pre-Treatment Via pH Adjustment

In one or more exemplary methods, the pH of the suspension (e.g. broth, fermentation broth, reaction milieu) can be adjusted prior to/during centrifuging.

In many cases utilizing fermentation broths, the pH of the suspension is in the range of 6-7. For example, the pH may be at 6.1, 6.2, 6.3, 6.4, 6.5, 6.6, 6.7, 6.8, or 6.9. However, other suspensions may have different starting pHs, such as below 6 and above 7. The particular starting pH of the suspension is not limiting.

Advantageously, reducing the pH of the suspension, therefore making it acidic, prior to centrifuging may improve sedimentation rate, and thus speed and yield processes. Further, a flocculation agent may be avoided as the pH adjustment may serve as an endogenous flocculation process.

The pH of the suspension may be adjusted through any number of methods. For example, an acid can be incorporated into the suspension to reduce the pH of the suspension. The acid can be an organic acid. In other embodiments, the acid can be an inorganic acid.

One example acid that can be used is sulfuric acid, such as having the formula H₂SO₄. For example, the acid could be 20% H₂SO₄-solution. However, other acids can be used as well, and the particular acid used is not limiting. For example, the acid can be selected from one or more of H₂SO₄, HCl, H₃PO₄, formic acid, acetic acid and citric acid. Each of these acids can be used alone or in combination with any other acids.

Through the pH adjustment, the suspension may be brought down to a pH of between 2 and 5, in particular between 3 and 4. For example, after the pH adjustment pre-treatment, the suspension may have a pH of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In certain implementations, after the pH adjustment pre-treatment, the suspension may have a pH of greater than 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9. In some implementations, after the pH adjustment pre-treatment, the suspension may have a pH of less than 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In one embodiment, after adjusting the pH of the suspension acidic, the pre-treated suspension is cooled down, e.g. to 5-15° C., and stored for 1-15 days before centrifuging.

Advantageously, the pH adjustment of the suspension can reduce a surface charge of particles (e.g., those of the biomass). Therefore, the pH adjustment can facilitate self-flocculation of the biomass, such as during centrifuging.

In one or more exemplary methods, the pH adjustment can be performed alone, with the dilution, with the heat treatment, or with both the heat treatment and the dilution.

2.2.2.2 Pre-Treatment Via Dilution

In one or more exemplary methods of the disclosed methods, the suspension (e.g. broth, fermentation broth, reaction milieu) can be diluted prior to/during centrifuging.

In certain embodiments, the suspension can be diluted so that the mass of the diluted suspension is between 1.1 and 10 times of the original mass of the suspension, preferably between 1.5 and 10, more preferably between 2 and 4, more preferably between 2.5 and 3.5. For example, the suspension may be diluted by 1.1, 1.2, 1.3, 1.4, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 times the original mass of the suspension. In some embodiments, the suspension may be diluted by less than 2.0, 2.5, 3.0, 3.5, 4.0, 4.5, 5.0, 5.5, 6.0, 6.5, 7.0, 7.5, 8.0, 8.5, 9.0, 9.5, or 10.0 times the original mass of the suspension. In some embodiments, the suspension may be diluted by greater than 1.5, 2.0, 2.5, 3.0, 3.5, 4.0, 4.5, 5.0, 5.5, 6.0, 6.5, 7.0, 7.5, 8.0, 8.5, 9.0, or 9.5 times the original mass of the suspension.

The dilution can be performed preferably with water. The water may be tap water, de-ionized water or distilled water.

In certain implementations, dilution can lower the density of supernatant solution and lower the viscosity. Another advantage of dilution is to increase the yield of the HMO product available in the supernatant. For example, without dilution, only around 50-70% of the HMO produced by fermentation go to the supernatant.

In certain implementations, after dilution the suspension may have a dilution ratio from about 2 to 10, preferably between 2 and 8, more preferably between 2 and 4, more preferably between 2.5 and 3.5. The dilution ratio is a ratio between the final volume of the suspension (i.e. after dilution) and the initial volume of the suspension (i.e. before dilution). For example, the suspension may be diluted by 2.0, 2.5, 3.0, 3.5, 4.0, 4.5, 5.0, 5.5, 6.0, 6.5, 7.0, 7.5, 8.0, 8.5, 9.0, 9.5, or 10.0 times the original volume of the suspension.

Preferably, the suspension may be diluted to achieve a particular bio-wet-mass (BWM). BWM is a ratio between the isolated wet solid pellet after centrifugation vs initial broth mass. To determine the BWM, a sample is taken from the stirred suspension and centrifuged with a relative centrifugal force (RCF) of around 10000 g until a clear supernatant is obtained (about 10 min). The supernatant is decanted and the mass of the solid wet pellet is weighted. BWM is the percent ratio of the solid wet pellet to the weight of the sample taken from the suspension.

Typical suspensions have a BWM of 25-35%, or all the way up to 50%, as determined under the conditions disclosed above. Thus, in some embodiments, the suspension may be diluted so that its BWM is less than 20, 15, 10 or 5%, preferably around 10-15% (the BWM after dilution is determined under identical conditions as before dilution). More preferably, this is achieved by a dilution with a dilution ratio of around 2.5-3.5, or so that the mass of the diluted suspension is between 2.5 and 3.5-fold of the original mass of the suspension. In some embodiments, the suspension may be diluted so that the BWM of the original suspension is reduced by 30-90%, for example the BWM is reduced to e.g. ⅔, ½, ⅓, ⅕ or 1/10 of the original BMW measured before dilution.

In some embodiments, dilution can be performed during a multiple-stage centrifugation, such as a 2-stage centrifugation. For example, the water may be added after the first centrifugation, but before the second centrifugation.

In one or more exemplary methods, the dilution can be performed alone, with the pH adjustment, with the heat treatment, or with both the heat treatment and the pH adjustment.

2.2.2.3 Pre-Treatment Via Heat Treatment

In one or more exemplary methods, the suspension can be heat treated (e.g. heat adjusted, temperature treated, change of temperature) prior to/during centrifuging.

In certain embodiments of the disclosure, the suspension can be pre-treated by heating the suspension up to a temperature of between 20 and 120° C., such as 30-90, 30-85 or 35-65° C., preferably 45 and 120° C., preferably between 60 and 90° C., more preferably between 60° C. and 80° C. For example, the suspension may be heated to a temperature of 20, 25, 30, 35, 40, 45, 50, 55, 60, 65, 70, 75, 80, 85, 90, 95, 100, 105, 110, 115 or 120° C. In some embodiments, the suspension may be heated to a temperature of greater than 20, 25, 30, 35, 40, 45, 50, 55, 60, 65, 70, 75, 80, 85, 90, 95, 100, 105, 110, 115 or 120° C. In some embodiments, the suspension may be heated to a temperature of less than 20, 25, 30, 35, 40, 45, 50, 55, 60, 65, 70, 75, 80, 85, 90, 95, 100, 105, 110, 115 or 120° C.

The heat treatment can be performed through known methods, such as the use of an oven, a burner, water bath, oil bath, heating mantle or other heating systems. Preferably, the heat treatment is performed by means of an industrial heater using one or a combination of the following heat transfer methods: conductive, convective, radiation. Such heaters can be e.g. circulation heaters, duck heaters, immersion heaters, heating with steam or heating through heating mantle.

In some embodiments, the suspension can be held at the heat treatment temperature for a particular time. For example, the suspension can be held at any of the above recited heating temperatures for 1, 2, 3, 4, 5, 10, 15, 20, 25 or 30 minutes. In some embodiments, the suspension can be held at any of the above recited heating temperatures for greater than 1, 2, 3, 4, 5, 10, 15, 20, 25 or 30 minutes. In some embodiments, the suspension can be held at any of the above recited heating temperatures for less than 2, 3, 4, 5, 10, 15, 20, 25 or 30 minutes.

In certain embodiments, the suspension is pre-treated by directly heating to any of the above-listed temperatures. In other implementations, the suspension can include stepwise heating, where the suspension can be heated to a first temperature for a first amount time. The suspension can then be heated to a second temperature for a second amount of time. The first temperature can be less than the second temperature. The first temperature can be greater than the second temperature (e.g. cooled instead of heated). The first amount of time can be greater than the second amount of time. The first amount can be less than the second amount of time. The first temperature can be any of the above-described temperatures. The second temperature can be any of the above-described temperatures. The first amount of time can be any of the above-described times. The second amount of time can be any of the above-described times.

Advantageously, the heat treatment can denature portions of the biomass, such as proteins and DNAs. Further, the heat treatment can precipitate biomolecules, such as proteins and DNAs. This can contribute to lowering the high initial broth viscosity.

In one or more exemplary methods, the heat treatment can be performed alone, with the dilution, with the pH adjustment, or with both the pH adjustment and the dilution.

2.2.2.4 Pre-Treatment Via pH Adjustment and Dilution

In one or more exemplary methods, both a pH adjustment and a dilution of the suspension may be performed. The combination of the two methodologies may provide synergistic effects that allow for a clearer supernatant and ease of centrifuging. For example, pH adjustment can reduce surface charge and when combined with dilution should provide for a better HMO yield.

In one or more exemplary methods, the pH adjustment of the suspension can be performed first, as discussed in detail above. Once the pH has been adjusted as desired, the suspension may be diluted to the desired level, as discussed in detail above. If the dilution affects the pH, the pH may be re-balanced in order to maintain the proper pH.

In one or more exemplary methods, the dilution of the suspension can be performed first, as detailed above. Once the suspension has been diluted to the desired level, the suspension can then undergo pH adjustment. Thus, the final diluted suspension may undergo pH adjustment which may require more acid to be used as compared to pH adjustment alone due to the higher volume of suspension.

In one or more exemplary methods, the dilution and the pH adjustment can occur at the same time (e.g. generally at the same time, concurrently, simultaneously). For example, water can be added into the suspension, and while this occurs acid may also be added. Alternatively, acid may be added first and water can be added at around the same time. In one or more exemplary methods, the pH adjustment and dilution can occur in repeating steps. For example, some acid may be added, then some water, which can be repeated until a desired pH and dilution is achieved.

Both the pH adjustment and dilution may occur prior to centrifuging or during centrifuging.

2.2.2.5 Pre-Treatment Via pH Adjustment and Heating

In one or more exemplary methods, both a pH adjustment and heating of the suspension may be performed. The combination of the two methodologies may provide synergistic effects that allow for a clearer supernatant and ease of centrifuging. For example, pH adjustment can reduce surface charge and when combined with denaturing of the heating a better HMO yield can be achieved.

In one or more exemplary methods, the pH adjustment of the suspension can be performed first as discussed in detail above. Once the pH has been adjusted as desired, the suspension may be heated to the desired temperature(s), as discussed in detail above. If the heating affects the pH, the pH may be re-balanced in order to maintain the proper dilution.

In one or more exemplary methods, the heating of the suspension can be performed first, as detailed above. Once the suspension has been heated to the desired temperature(s), the suspension can then undergo pH adjustment. The amount of acid required in combination with the heat treatment may change based on pH adjustment alone.

In one or more exemplary methods, the heating and the pH adjustment can occur at the same time (e.g. generally at the same time, concurrently, simultaneously). For example, a vessel containing the suspension may be heated in one or more of the methods discussed above. While the vessel is heating up, acid may be added to the suspension in order to adjust the pH of the suspension.

Both the pH adjustment and dilution may occur prior to centrifuging or during centrifuging.

2.2.2.6 Pre-Treatment Via Dilution and Heating

In one or more exemplary methods, both a dilution and heating of the suspension may be performed. The combination of the two methodologies may provide synergistic effects that allow for a clearer supernatant and ease of centrifuging. For example, the denaturing of proteins achieved by heating may allow for easier separation from dilution, which can lead to a better HMO yield.

In one or more exemplary methods, the dilution of the suspension can be performed first as discussed in detail above. Once the suspension has been diluted, the suspension may be heated to the desired temperature(s), as discussed in detail above. If the heating affects the dilution, such as the loss of material through evaporation, further water can be added in order to maintain the proper dilution.

In one or more exemplary methods, the heating of the suspension can be performed first, as detailed above. Once the suspension has been heated to the desired temperature(s), water can be added into the suspension. The water may be at the same temperature as the heated suspension, or may be at a lower or higher temperature.

In one or more exemplary methods, the heating and the dilution can occur at the same time (e.g. generally at the same time, concurrently, simultaneously). For example, a vessel containing the suspension may be heated in one or more of the methods discussed above. While the vessel is heating up, water may be added to the suspension in order to adjust the dilution of the suspension. Alternatively, dilution and heating can occur at the same time by adding hot water pre-heated to a temperature higher than the target temperature of the diluted suspension after adding into a cold suspension.

Both the heating and dilution may occur prior to centrifuging or during centrifuging.

2.2.2.7 Pre-Treatment Via Heating, pH Adjustment and Dilution

In one or more exemplary methods, all three pre-treatments of a dilution, a heating and a pH adjustment of the suspension may be performed. The combination of the three methodologies may provide synergistic effects that allow for a clearer supernatant and ease of centrifuging. Thus, higher yields at faster times can be achieved. The combination of the three pre-treatments can have a greater impact than the addition of all pre-treatments individually.

In one or more exemplary methods, the pH adjustment of the suspension can be performed first as discussed in detail above. Once the pH has been adjusted as desired, the suspension may be diluted to the desired level, as discussed in detail above. If the dilution affects the pH, the pH may be re-balanced in order to maintain the proper pH. Once the dilution has been performed, the suspension may be heated to the desired temperature(s), as discussed in detail above. If the heating affects the pH and/or the dilution, the pH and/or the dilution may be re-balanced in order to maintain the proper pH levels and/or dilution levels.

In one or more exemplary methods, the pH adjustment of the suspension can be performed first as discussed in detail above. Once the pH adjustment has been performed, the suspension may be heated to the desired temperature(s). If the heating affects the pH, the pH may be re-balanced in order to maintain the proper pH levels. Once the heating has been performed, the suspension may be diluted to the desired level. If the dilution affects the pH, the pH may be re-balanced in order to maintain the proper pH. The water (e.g., for dilution) may be at the same temperature as the heated suspension, may be at a lower temperature or at higher temperature.

In one or more exemplary methods, the heating of the suspension can be performed first, as detailed above. Once the suspension has been heated to the desired temperature(s), the suspension can then undergo pH adjustment. The amount of acid required in combination with the heat treatment may change based on pH adjustment alone. Once the heating and pH adjustment have been performed, the suspension may be further diluted. The water may be at the same temperature as the heated suspension, may be at a lower temperature or at higher temperature. If need be after dilution, the pH can be further adjusted to a desired level.

In one or more exemplary methods, the heating of the suspension can be performed first, as detailed above. Once the suspension has been heated to the desired temperature(s), the suspension can then undergo dilution. The water may be at the same temperature as the heated suspension, may be at a lower temperature or at higher temperature. Once the heating and dilution have been performed, the suspension may undergo pH adjustment. The amount of acid required in combination with the heat treatment and dilution may change based on pH adjustment alone.

In one or more exemplary methods, the dilution of the suspension can be performed first as discussed in detail above. Once the suspension has been diluted, the suspension may be heated to the desired temperature(s). If the heating affects the dilution, such as the loss of material through evaporation, further water can be added in order to maintain the proper dilution. After the heating, the suspension may undergo pH adjustment. The amount of acid required in combination with the heat treatment and dilution may change based on pH adjustment alone.

In one or more exemplary methods, the dilution of the suspension can be performed first as discussed in detail above. Once diluted, the suspension may undergo pH adjustment. The amount of acid required in combination with the heat dilution may change based on pH adjustment alone. After the pH adjustment, the suspension may be heated. Adjustments can be made to the suspension during the heating to maintain pH and dilution levels.

In one or more exemplary methods, the heating and the dilution can occur at the same time (e.g. generally at the same time, concurrently, simultaneously). For example, a vessel containing the suspension may be heated in one or more of the methods discussed above. While the vessel is heating up, water may be added to the suspension in order to adjust the dilution of the suspension. After the heating and dilution, the pH of the suspension then may be adjusted. Alternatively, the pH of the suspension may be adjusted prior to the concurrent heating and dilution.

In one or more exemplary methods, the heating and the pH adjustment can occur at the same time (e.g. generally at the same time, concurrently, simultaneously). For example, a vessel containing the suspension may be heated in one or more of the methods discussed above. While the vessel is heating up, acid may be added to the suspension in order to adjust the pH of the suspension. After the heating and the pH adjustment, the suspension may be diluted. Alternatively, the suspension may be diluted prior to a concurrent heating and pH adjustment.

In one or more exemplary methods, the dilution and the pH adjustment can occur at the same time (e.g. generally at the same time, concurrently, simultaneously). For example, water can be added into the suspension, and while this occurs acid may also be added. Alternatively, acid may be added first, and water can be added at around the same time. In one or more exemplary methods, the pH adjustment and dilution can occur in repeating steps. For example, some acid may be added, then some water, which can be repeated until a desired pH and dilution is achieved. After the dilution and the pH adjustment, the suspension may then be heated. Alternatively, the suspension may be heated prior to the concurrent dilution and pH adjustment.

In one or more exemplary methods, the dilution, the pH adjustment, and the heating can occur at the same time (e.g. generally at the same time, concurrently, simultaneously). Thus, all three pre-treatments can occur at the same time.

The heating, the pH adjustment, and the dilution may occur prior to centrifuging or during centrifuging.

2.2.3. Ultrafiltration (UF) of the Reaction Milieu or the Pre-Treated Reaction Milieu

The UF step of the claimed method comprises:
i) setting the pH of said reaction milieu to acidic, such as from around 3 to around 6, preferably not higher than around 5 and/or warming up said reaction milieu to a temperature that is higher than room (or ambient) temperature, preferably to about 30-90° C., more preferably to about 35-65° C., and
ii) contacting the reaction milieu obtained in step i) to an ultrafiltration (UF) membrane with a molecular weight cut-off (MWCO) of around 5-1000 kDa, such as 10-1000 kDa, wherein the membrane is preferably composed of a non-polymeric material.

The ultrafiltration step is to separate the biomass (or the remaining part of the biomass of the pre-treated reaction milieu) and, preferably, also high molecular weight suspended solids from the soluble components of the broth which soluble components pass through the ultrafiltration membrane in the permeate. This UF permeate (UFP) is an aqueous solution containing the produced sialylated HMO.

In a preferred embodiment, if the reaction milieu is centrifuged, microfiltered or filtered on a filter press or a drum filter, the UF membrane is composed of a non-polymeric material, more preferably a ceramic material.

In one embodiment, the pH of the reaction milieu or the centrifuged reaction milieu is set to acidic, such as from around 3 to around 6, preferably to a value that is not higher than around 5, more preferably to a value around 3-5. Setting the pH as disclosed above is especially advantageous, because it gives rise to a substantial reduction in the amount of dissolved biomolecules such as soluble proteins and DNAs due to a more effective denaturation and precipitation. The lower amount of dissolved biomolecules allows to use a higher MWCO UF membrane in the subsequent step that provides a better flux, a contributing factor to a higher productivity.

In other embodiment, the reaction milieu or the centrifuged reaction milieu is heated up to a temperature that is higher than an ambient temperature, i.e. room temperature or reaction milieu temperature, e.g. to a temperature within the range of from around 30 to around 90° C., preferably to about 35-85° C., e.g. to around 35-75° C., more preferably to around 50-75° C., such as around 60-65° C. This heat treatment before UF substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

Preferably, step i) above comprises setting the pH of said reaction milieu to not higher than 5 and warming up said reaction milieu to around 30-90° C., which particularly reduces protein solubility and thereby protein leakage into the UF permeate in the subsequent UF step.

In step ii) of the claimed method, a UF membrane composed of a non-polymeric material, preferably a ceramic membrane, is used. The non-polymeric UF membrane tolerates high temperature if UF is carried out at that temperature. Also, the applicable flux of a non-polymeric membrane, advantageously a ceramic membrane, is usually higher than that of a polymeric UF membrane with identical or similar MWCO; an addition, a non-polymeric membrane, advantageously a ceramic membrane, is less prone to fouling or getting clogged. In industrial application, the regeneration of the UF membrane is an important cost and technical factor. A non-polymeric membrane, advantageously a ceramic membrane, allows to use harsh clean-in-place (CIP) conditions including caustic/strong acid treatment at high temperature (not applicable on polymeric membranes), which may be required when a fermentation stream with high suspended solid content is ultrafiltered. Furthermore, a non-polymeric membrane, advantageously a ceramic membrane, has longer life-time due to inertness and abrasion-resistance to solid particles that circulate at high cross-flow.

Any conventional non-polymeric ultrafiltration membrane, advantageously ceramic membrane, can be used having a molecular weight cut-off (MWCO) range between about 5 and about 1000 kDa, such as around 5-250, 5-500, 5-750, 50-250, 50-500, 50-750, 100-250, 100-500, 100-750, 250-500, 250-750, 500-750 kDa, or any other suitable sub-ranges.

Step ii) of the method can be conducted at low temperature (around 5° C. to rt), at around room temperature or at elevated temperature, preferably at elevated temperature. The elevated temperature preferably does not exceed around 65° C.; suitable temperature ranges can be e.g. around 35-50, 35-65, 45-65, 50-65, 55-65 or 60-65° C. The UF step conducted at elevated temperature substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

Preferably, if step ii) is carried out at elevated temperature, it is advantageous to set the pH of the reaction milieu to not higher than around 5 in the preceding step i), because it particularly reduces protein solubility and thereby protein leakage into the UF permeate.

The ultrafiltration step can be applied in dead-end or cross-flow mode.

In one embodiment, only a single UF step is conducted in the method of the invention.

In other embodiment, the method of the invention may comprise more than one ultrafiltration steps using membranes with different MWCO, e.g. using two ultrafiltration separations wherein the first membrane has a higher MWCO than that of the second membrane, provided that at least one the UF membranes is a non-polymeric membrane, preferably a ceramic membrane. This arrangement may provide a better separation efficacy of the higher molecular weight components of the broth.

Yet in one embodiment, the ultrafiltration can be combined with diafiltration.

After conducting ultrafiltration comprising steps i) and ii) disclosed above, the UF permeate contains materials that have a molecular weight lower than the MWCO of the utilized membrane, optionally the MWCO of the last membrane when a series of membranes are used, including sialylated HMO of interest.

2.2.4. Centrifugation

After any or all of the above pre-treatments, the biomass is separated from the suspension by centrifugation. It can have advantages over other processes such as ultrafiltration, which can take a significant amount of time. The centrifuging can be lab scale or, advantageously over previous centrifuging methods, commercial scale (e.g. industrial scale, full production scale).

In some embodiments, a multi-step centrifugation can be used. For example, a series of 2, 3, 4, 5, 6, 7, 8, 9 or 10 centrifugation steps can be performed. In other implementations, the centrifugation may be a single step.

Centrifugation provides a quick biomass-removal compared to lengthy ultrafiltration both on lab and full production scale with a long clean-in-place (CIP) procedure, requirement of expensive ceramic membranes and high size/high foot-print area.

Biomass separation is then achieved by a more efficient and economical way compared to ultrafiltration. The current biomass removal by industrial continuous ultrafiltration has low permeate flow rate, that requires high residence time (>1 h), high membrane area associated with equipment high foot-print area, high energy consumption by powerful cross-flow pumps, has performance that is broth-dependent and sometime unpredictable, requires long CIP (5 h) every 24 h of operation or less, has a CapEx which is very high both for UF unit and ceramic membranes, and has a high size/footprint.

In certain embodiments, Sedicanter® centrifuge designed and manufactured by Flottweg can be used.

The particular type of centrifuge is not limiting, and many types of centrifuges can be used. The centrifuging can be a continuous process. In some embodiments, the centrifuging can have feed addition. For example, the centrifuging can have a continuous feed addition. In certain embodiments, the centrifuging can include a solid removal, such as a wet solid removal. The wet solid removal can be continuous in some implementations, and periodic in other implementations.

For example, a conical plate centrifuge (e.g. disk bowl centrifuge or disc stack separator) can be used. The conical plate centrifuge can be used to remove solids (usually impurities) from liquids or to separate two liquid phases from each other by means of a high centrifugal force. The denser solids or liquids which are subjected to these forces move outwards towards the rotating bowl wall while the less dense fluids move towards the centre. The special plates (known as disc stacks) increase the surface settling area which speeds up the separation process. Different stack designs, arrangements and shapes are used for different processes depending on the type of feed present. The concentrated denser solid or liquid can then be removed continuously, manually or intermittently, depending on the design of the conical plate centrifuge. This centrifuge is very suitable for clarifying liquids that have small proportion of suspended solids.

The centrifuge works by using the inclined plate setter principle. A set of parallel plates with a tilt angle θ with respect to horizontal plane is installed to reduce the distance of the particle settling. The reason for the tilted angle is to allow the settled solids on the plates to slide down by centrifugal force so they do not accumulate and clog the channel formed between adjacent plates.

This type of centrifuge can come in different designs, such as nozzle-type, manual-cleaning, self-cleaning, and hermetic. The particular centrifuge is not limiting.

Factors affecting the centrifuge include disk angle, effect of g-force, disk spacing, feed solids, cone angle for discharge, discharge frequency, and liquid discharge.

Alternatively, a solid bowl centrifuge (e.g. a decanter centrifuge) can be used. This is a type of centrifuge that uses the principle of sedimentation. A centrifuge is used to separate a mixture that consists of two substances with different densities by using the centrifugal force resulting from continuous rotation. It is normally used to separate solid-liquid, liquid-liquid, and solid-solid mixtures. One advantage of solid bowl centrifuges for industrial uses is the simplicity of installation compared to other types of centrifuge. There are three design types of solid bowl centrifuge, which are conical, cylindrical, and conical-cylindrical.

Solid bowl centrifuges can have a number of different designs, any of which can be used for the disclosed method. For example, conical solid bowl centrifuges, cylindrical solid bowl centrifuges, and conical-cylindrical bowl centrifuges can be used.

With the help of helical screw conveyor, solid bowl centrifuges separate two substances with different densities by the centrifugal force formed under fast rotation. Feed slurry enters the conveyor and is delivered into the rotating bowl through discharge ports. There is a slight speed difference between the rotation of conveyor and bowl, causing the solids to convey from the stationary zone where the wastewater is introduced to the bowl wall. By centrifugal force, the collected solids move along the bowl wall, out of the pool and up the dewatering beach located at the tapered end of the bowl. At last the solids separated go to solid discharge while the liquids go to liquid discharge. The clarified liquid flows through the conveyor in the opposite direction through adjustable overflow parts.

The centrifuging can be performed at a number of speeds and residence times. For example, the centrifuging can be performed with a relative centrifugal force (RCF) of 20000 g, 15000 g, 10000 g, or 5000 g. In some embodiments, the centrifuging can be performed with a relative centrifugal force (RCF) of less than 20000 g, 15000 g, 10000 g or 5000 g. In some embodiments, the centrifuging can be performed with a relative centrifugal force (RCF) of greater than 20000 g, 15000 g, 10000 g or 5000 g.

In some embodiments, the centrifuging can be characterized by working volume. In some embodiments, the working volume can be 1, 5, 10, 15, 20, 50, 100, 300 or 500 litre (1). In some embodiments, the working volume can be less than 1, 5, 10, 15, 20, 50, 100, 300 or 500 l. In some embodiments, the working volume can be greater than 1, 5, 10, 15, 20, 50, 100, 300 or 500 l.

In some embodiments, the centrifuging can be characterized by feed flow rate. In some embodiments, the feed flow rate can be 100, 500, 1000, 1500, 2000, 5000, 10000, 20000, 40000 or 100000 l/hr. In some embodiments, the feed flow rate can be greater than 100, 500, 1000, 1500, 2000, 5000, 10000, 20000, 40000 or 100000 l/hr. In some embodiments, the feed flow rate can be less than 100, 500, 1000, 1500, 2000, 5000, 10000, 20000, 40000 or 100000 l/hr.

The amount of time spent centrifuging (e.g., residence time) can vary as well. For example, the residence time can be 0.1, 0.2, 0.5, 1, 2, 3, 4, 5, 6, 7, 8, 9 or 10 minutes. In some embodiments, the residence time can be greater than 0.1, 0.2, 0.5, 1, 2, 3, 4, 5, 6, 7, 8, 9 or 10 minutes. In some embodiments, the residence time can be less than 0.1, 0.2, 0.5, 1, 2, 3, 4, 5, 6, 7, 8, 9 or 10 minutes.

2.2.4.1 Supernatant

The above described centrifugation processes can allow for the formation of an improved supernatant. For example, the supernatant can have the sialylated HMOs, while the remaining material can be separated out. Thus, the supernatant can be a clear (e.g. clarified, generally clear, mostly clear) supernatant. The remaining material (which can cause a lack of clarity of the supernatant) can be, for example, a biomass, that is one or more of cells, cell fragments, small particles, and biomolecules. The biomolecules can be, for example, proteins, RNAs and DNAs. Thus, the supernatant can be a clarified supernatant containing purified sialylated HMOs.

As an example of clarity for a supernatant, a typical fermentation broth can have very high optical density at 600 nm (OD600), more than 10 relative to pure water. OD600 is usually measured with a spectrophotometer; dilution of the sample may be necessary if OD600 goes above 1 due to high amount of suspended particles (and thus the OD600 value is calculated with respect to the dilution). However, after the above-recited processes, the OD600 can move to below 1.0, 0.7, 0.5, 0.3, 0.2, 0.1 or 0.05, which is measured without dilution but relative to micro-filtered sample of the obtained supernatant. In some embodiments, the OD600 can move to above 1.0, 0.7, 0.5, 0.3, 0.2, 0.1 or 0.05. In some embodiments, the OD600 can move to 1.0, 0.7, 0.5, 0.3, 0.2, 0.1 or 0.05. When OD600 is less than 0.1, the suspended particles cannot be visually detected and the supernatant would look as a clear solution.

Further, the proteins in the supernatant can be reduced as well, providing further clarity. The proteins in the supernatant post centrifuging can be less than 3000, 2500, 2000, 1500, 1000, 500, 400, 300, 200, 100 or 50 mg/l. In some embodiments, the proteins in the supernatant post centrifuging can be greater than 3000, 2500, 2000, 1500, 1000, 500, 400, 300, 200, 100 or 50 mg/l. In some embodiments, the proteins in the supernatant post centrifuging can be 3000, 2500, 2000, 1500, 1000, 500, 400, 300, 200, 100 or 50 mg/l.

Further, advantageously embodiments of the disclosed methods can provide for a higher product yield as compared to previous methods. The clarified supernatant can have a product yield of 50, 55, 60, 65, 70, 75, 80, 85, 90 or 95%. The clarified supernatant can have a product yield of >50, 55, 60, 65, 70, 75, 80, 85, 90 or 95%. The clarified supernatant can have a product yield of <50, 55, 60, 65, 70, 75, 80, 85, 90 or 95%.

Any of the above supernatant properties can be produced through a single instance of centrifuging. Alternatively, it can be produced through multiple instances of centrifuging.

2.2.5 Post-Processing for Purification

After producing the above-described UF permeate (2.2.3) or CF supernatant (2.2.4.1), further post-processing steps can be taken. This can be done to, for example, isolate (e.g. obtain) sialylated HMOs from the UF permeate or supernatant. A number of different methods are discussed below. It will be understood that the method can include any one of the disclosed post-processing steps. In alternative embodiments, the method can contain more than one of the disclosed post-processing steps, in any configuration of the steps.

2.2.5.1 UF after Centrifugation

In some embodiments, the post-processing method can include ultrafiltration (UF) or microfiltration (MF) of the clarified supernatant obtained after centrifugation. For example, the filtration can use a membrane having a pore size <2 m and >2 nm and/or MWCO >1000 Da or 10000 Da or above. The pore size or cut-of should be large enough to allow the product (sialylated HMO) to pass though the pores of the membrane. Optionally, there can further be diafiltration, wherein the sialylated HMOs are collected in a permeate stream.

In an optional post-processing step, ultrafiltration is to separate the remaining part of the biomass after centrifugation. The UF (or MF) membrane could be composed of a polymeric or non-polymeric material. Preferably, the UF membrane is composed of a non-polymeric material, more preferably a ceramic material. The non-polymeric UF membrane tolerates high temperature if UF is carried out at that temperature. Also, the applicable flux of a non-polymeric membrane, advantageously a ceramic membrane, is usually higher than that of a polymeric UF membrane with identical or similar MWCO; an addition, a non-polymeric membrane, advantageously a ceramic membrane, is less prone to fouling or getting clogged. In industrial application, the regeneration of the UF membrane is an important cost and technical factor. A non-polymeric membrane, advantageously a ceramic membrane, allows to use harsh clean-in-place (CIP) conditions including caustic/strong acid treatment at high temperature (not applicable on polymeric membranes), which may be required when a fermentation stream with high suspended solid content is ultrafiltered. Furthermore, a non-polymeric membrane, advantageously a ceramic membrane, has longer life-time due to inertness and abrasion-resistance to solid particles that circulate at high cross-flow.

Any conventional ultrafiltration membrane, advantageously ceramic membrane, can be used having a molecular weight cut-off (MWCO) range between about 1 and about 1000 kDa, such as around 1-10, 10-1000, 5-250, 5-500, 5-750, 50-250, 50-500, 50-750, 100-250, 100-500, 100-750, 250-500, 250-750, 500-750 kDa or any other suitable sub-ranges.

UF can be conducted at low temperature (around 5° C. to rt), at around room temperature or at elevated temperature, preferably at elevated temperature. The elevated temperature preferably does not exceed around 80° C., suitable temperature ranges can be e.g. around 35-50, 35-80, 45-65, 50-65, 55-65 or 60-80° C. The UF step conducted at elevated temperature substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

The ultrafiltration step can be applied in dead-end or cross-flow mode. Yet in one embodiment, the ultrafiltration can be combined with diafiltration. The sialylated HMO is collected in the UF permeate (UFP).

2.2.5.2 Nanofiltration

The method of the invention comprises, as optional step, a nanofiltration (NF) step. Preferably, the NF step directly follows the ultrafiltration of the reaction milieu (2.2.3) or the centrifuged reaction milieu (2.2.5.1), that is the feed of the NF step is the UF permeate containing the sialylated HMO of interest. Also preferably, the NF step directly follows the centrifugation step (2.2.4). Optionally, the UF permeate or the clarified supernatant can be decolorized by using active charcoal (see below) before conducting the NF step. This nanofiltration step may advantageously be used to concentrate the UF permeate or the clarified supernatant, and/or to remove ions, mainly monovalent ions, and organic materials having a molecular weight lower than that of the sialylated HMO, such as monosaccharides. The nanofiltration membrane has a lower MWCO than that of the ultrafiltration membrane(s) used in the precedent step and ensures the retention of the sialylated HMO of interest.

In a first aspect of nanofiltration, the MWCO of the NF membrane is around 25-50% of the molecular weight of the sialylated HMO of interest, typically around 150-500 Da. In this regard, the sialylated HMO of interest is accumulated in the NF retentate (NFR). The nanofiltration can be combined with diafiltration with water to remove, or to reduce the amount of, permeable salts such as monovalent ions more effectively.

In one embodiment of the first aspect, NF follows UF so that the UF permeate is nanofiltered without diafiltration, and the NF retentate containing the sialylated HMO is collected and subjected to further separation step(s) of the method.

In other embodiment of the first aspect, NF follows UF so that the UF permeate is nanofiltered followed by diafiltration, and the NF retentate containing the sialylated HMO is collected and subjected to further separation step(s) of the method.

The first aspect of the nanofiltration is advantageously applicable for UF permeate that does not contain lactose or contains only minor amount of lactose (at most around 1-2% of the weight of the sialylated HMO of interest).

In the second aspect of nanofiltration, which is advantageously applicable for UF permeate that contains more lactose, the membrane has a MWCO of 600-3500 Da ensuring the retention of the tri- or higher sialylated HMO and allowing at least a part of lactose to pass through the membrane, wherein the active (top) layer of the membrane is composed of polyamide, and wherein the MgSO₄ rejection factor on said membrane is around 20-90%, preferably 50-90%.

The term “ensuring the retention of the tri- or higher sialylated HMO” preferably means that, during the nanofiltration step, the tri- or higher sialylated HMOs do not pass, or at least significantly do not pass, through the membrane and thus their vast majority will be present in the retentate. The term “allowing at least a part of lactose to pass through the membrane” preferably means, that lactose, at least partially, can penetrate the membrane and be collected in the permeate. In case of high rejection (about 90%) of lactose, a subsequent diafiltration with pure water may be necessary to bring all or at least the majority of the lactose in the permeate. The higher the lactose rejection the more diafiltration water is necessary for efficient separation.

The applied nanofiltration membrane according to the second aspect of NF shall be tight for tri- and higher sialylated HMOs in order that they are efficiently retained. Preferably, the rejection of the tri- or higher sialylated HMO is more than 95%, more preferably 97%, even more preferably 99%. Membranes with MWCO of more than 3500 Da are expected to allow more or significant amount of tri- or higher sialylated HMOs pass through the membrane thus show a reduced retention of tri- or higher sialylated HMO and therefore are not suitable for the purposes of the invention, and can be excluded. It is preferred that the rejection of the lactose is not more than 80-90%. If the lactose rejection turns to be 90±1-2%, the tri- or tetrasaccharide sialylated HMO rejection shall preferably be around 99% or higher in order to achieve a practically satisfying separation.

The above requirements are simultaneously fulfilled when the membrane is relatively loose for MgSO₄, that is its rejection is about 50-90%. In this regard the above specified membrane is tight for tri- and higher sialylated HMOs, and loose for monosaccharides and lactose, and as well as for MgSO₄. Therefore, it is possible to separate lactose, which is a precursor in making human milk oligosaccharides enzymatically or by fermentation, from the sialylated human milk oligosaccharides product by nanofiltration with a good efficacy, and additionally a substantial part of divalent ions also passes to the permeate. In some embodiments, the MgSO₄ rejection factor is 30-90%, 20-80%, 40-90%, 40-80%, 60-90%, 70-90%, 50-80%, 50-70%, 60-70% or 70-80%. Preferably, the MgSO₄ rejection factor on said membrane is 80-90%. Also preferably, the membrane has a rejection factor for NaCl that is lower than that for MgSO₄. In one embodiment, the rejection factor for NaCl is not more than around 50%. In other embodiment, the rejection factor for NaCl is not more than around 40%. In other embodiment, the rejection factor for NaCl is not more than around 30%. In other embodiment, the rejection factor for NaCl is not more than around 20%. At a NaCl rejection of around 20-30%, a substantial reduction of all monovalent salts in the retentate is also achievable.

Also preferably, in some embodiments, the pure water flux of the membrane is at least 50 l/m² h (when measured at 23-25° C., 10 bar and constant cross-flow of 300 l/h). Preferably, the pure water flux of the membrane is at least 60 l/m² h, at least 70 l/m² h, at least 80 l/m² h or at least 90 l/m² h.

The active or the top layer of nanofiltration membrane suitable in the second aspect of the NF step is preferably made of polyamide. Although membranes of different type seem to have promising separation efficacy, for example NTR-7450 having sulphonated PES as active layer for separating lactose and 3′-SL (Luo et al. (Biores. Technol. 166, 9 (2014); Nordvang et al. (Separ. Purif Technol. 138, 77 (2014)), the above specified membrane used in the invention shows always better separation of lactose from a sialylated HMO. In addition, the above mentioned NTR-7450 membrane is subject to fouling, which typically results in a drop in flux, increasing the lactose rejection and therefore a reduced separation factor. Yet preferably, the polyamide membrane is a polyamide with phenylene diamine or piperazine building blocks as amine, more preferably piperazine (referred to as piperazine-based polyamide, too).

Yet preferably, the membrane suitable for the purpose of the present invention is a thin-film composite (TFC) membrane.

An example of suitable piperazine based polyamide TFC membranes is TriSep® UA60.

The nanofiltration membrane suitable in the second aspect of the NF step is characterized by some or all of the above features and thus one or more of the following benefits are provided: selectively and efficiently removes lactose, from tri- or higher sialylated HMOs yielding an enriched tri- or higher sialylated HMO fraction; removes efficiently monovalent as well as divalent salts therefore no ion exchange step may be necessary or, if desalination is still needed, the ion exchange treatment requires substantially less resin; higher flux during the nanofiltration can be maintained compared to other membranes used for the same or similar purpose in the prior art, which reduces the operation time; the membrane disclosed above is less prone to getting clogged compared to the prior art solutions; the membrane disclosed above can be cleaned and regenerated completely therefore can be recycled without substantial reduction of its performance.

In one embodiment of the second aspect of the NF step, the step comprises:
contacting the UF permeate with a nanofiltration membrane with a molecular weight cut-off (MWCO) of 600-3500 Da, preferably around 1000 Da, ensuring the retention of the tri- or higher sialylated HMO and allowing at least a part of the lactose to pass through the membrane, wherein the active (top) layer of the membrane is composed of polyamide, and wherein the MgSO₄ rejection factor on said membrane is around 50-90
a subsequent optional diafiltration with said membrane,
and collecting the retentate enriched in the tri- or higher sialylated HMO.

In other embodiment, the NF step comprises:
contacting the UF permeate with a piperazine-based polyamide nanofiltration membrane with a molecular weight cut-off (MWCO) of around 1000 Da ensuring the retention of the tri- or higher sialylated HMO and allowing at least a part of the lactose to pass through the membrane, wherein the MgSO₄ rejection factor on said membrane is around 80-90%, and wherein
   the NaCl rejection factor on said membrane is lower than that for MgSO₄, and/or
   the pure water flux value of said membrane is at least around 50 l/m² h,
a subsequent optional diafiltration with said membrane,
and collecting the retentate enriched in the tri- or higher sialylated HMO.

Preferably, the NaCl rejection factor of the membrane is at most the half of the MgSO₄ rejection factor.

To achieve all the benefits mentioned above, the nanofiltration membrane to be applied in the second aspect of the NF step, preferably:
is a piperazine-based polyamide membrane with a MWCO of around 1000 Da,
has a MgSO₄ rejection of around 50-90%, preferably 80-90%,
has a NaCl rejection of not more than around 30%, and
has a pure water flux value of at least around 50 l/m² h, preferably around 90 l/m² h.

Also in a preferred embodiment, the separation factor of lactose over a sialylated human milk oligosaccharide is more than 10, preferably more than 25, more preferably more than 50, even more preferably more than 100.

Yet preferably, the separation factor of lactose over 3′-SL or 6′-SL is more than 20, preferably more than 50.

Yet preferably, the separation factor of lactose over a tetra- or pentasaccharide containing a sialyl moiety is more than 25, preferably more than 50, more preferably more than 100.

The second aspect of the NF step can be conducted under conditions used for conventional nanofiltration with tangential flow or cross-flow filtration with positive pressure compared to permeate side followed by diafiltration where both operations could be performed in a batch mode or preferably in continuous mode. The optional diafiltration, in one embodiment, is conducted by adding pure water to the retentate after the nanofiltration step disclosed above and continuing the filtration process with constant removal of permeate under the same or similar conditions as nanofiltration. The preferred mode of water addition is continuous, i.e. the addition flow rate is matching approximately the permeate flow rate. NF could be performed in a batch mode where retentate stream is recycled back to the feed tank and the diafiltration (DF) is done by adding purified or deionized water to the feed tank continuously. Most preferably, DF water is added after at least some pre-concentration by removing a certain amount of permeate. The higher concentration factor before the start of DF the better DF efficacy is achieved. After completion of DF, further concentration could be achieved by removing extra amount of permeate. Alternatively, NF could be performed in continuous mode preferably in multi-loop system where retentate from each loop is transferred to the next loop. In this case, DF water could be added separately in each loop with the flow rate either matching the permeate flow rate in each loop or at a lower flow rate. Like in batch mode DF, to improve the efficacy of DF, less water or no water should be added in the e.g. first loop to achieve a higher concentration factor. The distribution of water in multi-loop system as well as other process parameters such as transmembrane pressure, temperature and cross-flow is subject to routine optimization. The optional diafiltration, in other embodiment, can be performed with an aqueous solution of an inorganic electrolyte, according to WO 2019/003135. The inorganic electrolyte implies an inorganic salt that is at least partially soluble, preferably well soluble in water. The rejection of the inorganic salt on the membrane used in the method of invention shall not be higher than that of MgSO₄. In one embodiment, the aqueous solution of the inorganic electrolyte (salt) is neutral, that is a salt of a strong inorganic acid and a strong inorganic base. Exemplary embodiments of strong inorganic acids are HCl, HBr, sulfuric acid, nitric acid, phosphoric acid and perchloric acid, and those of strong inorganic bases are NaOH, KOH, LiOH, Mg(OH)₂ and Ca(OH)₂; the salts formed from these acid and bases are comprised in the group of neutral inorganic electrolytes applicable for the purpose of the invention. In other embodiment, the aqueous solution of the inorganic electrolyte is slightly basic, this is the case when a strong inorganic base (see above) forms a salt with a weak inorganic acid, e.g. carbonic acid (making carbonates) or acetic acid (making acetates). The inorganic electrolyte, usually, is an univalent or a bivalent anion electrolyte. Univalent anion electrolytes are e.g. the salts of HCl, HBr, nitric acid or perchloric acid, particularly chlorides such as LiCl, NaCl, KCl, MgCl₂, CaCl₂, or bicarbonates such as NaHCO₃. Divalent anion electrolytes are e.g. the salts of sulfuric acid such as Na₂SO₄, K₂SO₄, MgSO₄, Na₂SO₄, or carbonates such as Na₂CO₃, K₂CO₃. Especially preferred inorganic electrolyte is NaCl. Preferably, the aqueous solution of an inorganic electrolyte used in the diafiltration is a single electrolyte, that is the aqueous solution contains one and only electrolyte (salt).

The pH of the feed solution applied for second aspect of the NF step is, preferably, not higher than around 7, more preferably between around 3 and 7, even more preferably around 4 and 5, or around 5 and 6. A pH that is lower than 3 may adversely influence the membrane and the solute properties.

The convenient temperature range applied for second aspect of the NF step is between around 10 and around 60° C. Higher temperature provides a higher flux and thus accelerates the process. The membrane is expected to be more open for flow-through at higher temperatures, however this doesn't change the separation factors significantly. A preferred temperature range for conducting the nanofiltration separation according to the invention is around 20-45° C.

A preferred applied pressure in the nanofiltration separation is about 2-50 bars, such as around 10-40 bars. Generally, the higher the pressure the higher the flux.

In certain embodiments, the method of the invention may comprise additional (one or more) NF steps, preferably following an active charcoal treatment (see below) and/or ion exchange treatment (see below), wherein the main purpose is to concentrate the aqueous solution containing the sialylated HMO of interest.

2.2.5.3 Treatment with an Ion Exchange Resin

The aqueous solution of the sialylated HMO obtained as clarified supernatant as disclosed above (2.2.4.1), UF permeate disclosed above (2.2.3 or 2.2.5.1), NF retentate disclosed above (2.2.5.2) may optionally be further purified by means of an ion exchange resin. Any the UF permeate and the NF retentate can optionally be decolorized by using active charcoal (see below) before the treatment with ion exchange resin. Residual salts, colour bodies, biomolecules that contains ionizable groups (such as proteins, peptides, DNA and endotoxins), sialylated or zwitter-ionic compounds containing ionizable functional groups (such as amino groups in metabolites including biogenic amines, amino acids), compounds with acid-containing groups (such as organic acids, amino acids) can be further removed by treatment with resin. Especially, low salt content of the resin eluate (demineralization) can be achieved if a cation and an anion exchange resin is applied in the “treatment with an ion exchange resin”.

According to one embodiment, the ion exchange resin is a cation exchange resin, preferably a strongly acidic cation exchange resin, preferably in protonated form. In this step, the positively charged materials can be removed from the feed solution as they bind to the resin. The solution of the sialylated HMO is contacted with the cation exchange resin in any suitable manner which would allow positively charged materials to be adsorbed onto the cation exchange resin, and the sialylated HMO to pass through. The resulting liquid, after contacting with the cation exchange resin, contains the sialylated HMO besides anions in the form of the corresponding acids and carbohydrates like lactose (if still left after one or more previous purification steps). The acids can be neutralized conventionally.

In one embodiment, both cation and anion exchange resin chromatography, in any order, can be applied. If both cation and anion resin treatment are applied, the cation exchange resin is a strong acidic resin and an anion exchange resin is a weak anion resin in free base form. The cation exchange resin may be in H⁺-form or salt form, preferably in H⁺-form. This particular arrangement, besides removing salts and charged molecules from the remaining culture medium, can physically adsorb proteins, DNA and colorizing/caramel bodies efficiently that were left in the culture medium.

The application of a weak basic anion exchanger in free base form (that is, wherein the resin's functional group is a primary, secondary or tertiary amine) is advantageous compared to that of a strong basic anion exchanger in OH⁻-form. The strong basic exchanger has the ability, due to its strong basicity, to deprotonate the anomeric OH-group of the sialylated HMO. This initiates rearrangement reactions in the structure of the sialylated HMO and thus creates by-products, and/or a significant amount of sialylated HMO is bound to the resin. Consequently, both events contribute to the reduction of the recovery yield of the sialylated HMO.

In one embodiment, the ion exchange resin treatment step of the claimed method consists of the treatment of the aqueous solution of the sialylated HMO (which aqueous solution is an UF permeate as disclosed above, which can optionally be decolorized with active charcoal, or a NF retentate as disclosed above, which can optionally be decolorized with active charcoal, or a clarified centrifuge supernatant as disclosed above, which can optionally be decolorized with active charcoal) with a strong cation exchange resin in H⁺-form directly followed by a treatment with a weak anion exchange resin in free base form.

In other embodiment, the claimed method for obtaining or isolating a sialylated HMO from the reaction milieu in which they have been produced comprises only a single ion exchange resin treatment step with a strong cation exchange resin in H⁺-form.

When using an ion exchange resin, its degree of crosslinking can be chosen depending on the operating conditions of the ion exchange column. A highly crosslinked resin offers the advantage of durability and a high degree of mechanical integrity, however suffers from a decreased porosity and a drop off in mass-transfer. A low-crosslinked resin is more fragile and tends to swell by absorption of mobile phase. The particle size of the ion exchange resin is selected to allow an efficient flow of the eluent, while the charged materials are still effectively removed. A suitable flow rate may also be obtained by applying a negative pressure to the eluting end of the column or a positive pressure to the loading end of the column, and collecting the eluent. A combination of both positive and negative pressure may also be used. The ion exchange treatment can be carried out in a conventional manner, e.g. batch-wise or continuously.

Non-limiting examples of a suitable acidic cation exchange resin can be e.g. Amberlite IR100, Amberlite IR120, Amberlite FPC22, Dowex 50WX, Finex CS16GC, Finex CS13GC, Finex CS12GC, Finex CS11GC, Lewatit S, Diaion S K, Diaion U B K, Amberjet 1000, Amberjet 1200, Dowex 88.

Non-limiting examples of a suitable basic anion exchange resin can be e.g. Amberlite IRA67, Amberlite IRA 96, Amberlite IRA743, Amberlite FPA53, Diaion CRB03, Diaion WA10, Dowex 66, Dowex Marathon, Lewatit MP64.

2.2.5.4 Active Charcoal Treatment

According to certain embodiments, the method of invention comprises the optional step of active charcoal treatment. The optional active charcoal treatment may follow any of centrifugation (2.2.4), UF step (2.2.3 or 2.2.5.1), NF step (2.2.5.2) or the ion exchange treatment step (2.2.5.3), all disclosed above. The active charcoal treatment helps to remove colorizing agents and/or to reduce the amount of water soluble contaminants, such as salts, if required. Moreover, the active charcoal treatment removes residual or trace protein, DNA or endotoxin that may remain incidentally after the previous steps.

A carbohydrate substance like a sialylated HMO of interest tends to bind to the surface of charcoal particles from its aqueous solution, e.g. an aqueous solution obtained after the UF step, the NF step or the ion exchange treatment step. Similarly, the colorizing agents are also capable to adsorb to the charcoal. While the carbohydrates and colour giving materials are adsorbed, water soluble materials that are not or are more weakly bound to the charcoal can be eluted with water. By changing the eluent from water to aqueous alcohol, e.g. ethanol, the adsorbed sialylated HMO can easily be eluted and collected in a separate fraction. The adsorbed colour giving substances would still remain adsorbed on the charcoal, thus both decolourization and partial desalination can be achieved simultaneously in this optional step. However, due to the presence of organic solvent (ethanol) in the elution solvent, the efficacy of decolorization is lower compared to the case when the elution is done with pure water (see below).

Under certain conditions, the sialylated HMO is not, or at least not substantially, adsorbed to the charcoal particles and elution with water gives rise to an aqueous solution of the sialylated HMO without a significant loss in its amount, while the colour giving substances remain adsorbed. In this case, there is no need to use organic solvent such as ethanol for elution. It is a matter of routine skills to determine the conditions under which the sialylated HMO would bind to the charcoal from its aqueous solution. For example, in one embodiment, a more diluted solution of the sialylated HMO or a higher amount of charcoal relative to the amount of the sialylated HMO is used, in another embodiment a more concentrated solution of the sialylated HMO and a lower amount of charcoal relative to the amount of the sialylated HMO is applied.

The charcoal treatment can be conducted by adding charcoal powder to the aqueous solution of the sialylated HMO under stirring, filtering off the charcoal, re-suspending in aqueous ethanol under stirring and separating the charcoal by filtration. In higher scale purification, the aqueous solution of sialylated HMO after is preferably loaded to a column packed with charcoal, which may optionally be mixed with celite, then the column is washed with the required eluent. The fractions containing the sialylated HMO are collected. Residual alcohol, if used for elution, may be removed from these fractions by e.g. evaporation, to give an aqueous solution of the sialylated HMO.

Preferably, the active charcoal used is granulated. This ensures a convenient flow-rate without applying pressure.

Also preferably, the active charcoal treatment, more preferably the active charcoal chromatography, of the aqueous solution containing the sialylated HMO of interest from the UF step, NF step or the ion exchange treatment step, is conducted at elevated temperature. At elevated temperature, the binding of the salts, colour bodies, residual proteins, etc. to the charcoal particles takes place in a shorter contact time, therefore the flow-rate can be conveniently raised. Moreover, the active charcoal treatment conducted at elevated temperature substantially reduces the total number of viable microorganisms (total microbial count) in the aqueous solution of the sialylated HMO, thus a sterile filtration step in the later phase of the method may not be necessary. The elevated temperature is at least 30-35° C., such as at least 40° C., at least 50° C., around 40-50° C. or around 60° C.

Also preferably, the amount the applied charcoal is not more than around 10 weight % of the sialylated HMO contained in the load, more preferably around 2-6 weight %. This is economical, because all the benefits disclosed above can be conveniently achieved with a very low amount of charcoal.

It is especially preferred when the active charcoal treatment, preferably chromatography, is conducted at elevated temperature with not more than around 10 weight %, preferably around 2-6 weight %, of active charcoal relative to the amount of the sialylated HMO of interest in the aqueous solution.

2.2.5.5 Providing the Sialylated HMO of Interest in Isolated Form

After the separation/purification steps disclosed in any of 2.2.3 to 2.2.5.4 above, the sialylated HMO of interest so-obtained can be provided in its solid form by means of spray-drying or freeze-drying. Accordingly, the method of the invention may comprise one or more further steps of providing the sialylated HMO of interest in isolated, preferably, dried form, such as a step of spray-drying an aqueous solution of the sialylated HMO obtained after centrifugation, the UF step, NF step, active charcoal treatment and/or ion exchange treatment; or a step of freeze-drying an aqueous solution of the sialylated HMO obtained after centrifugation, the UF step, NF step, active charcoal treatment and/or ion exchange treatment. Alternatively, the sialylated HMO obtained after the UF step, NF step, active charcoal treatment and/or ion exchange treatment may be provided in a form of a concentrated aqueous solution or syrup by removing water, e.g. by means of distillation, preferably vacuum distillation, or nanofiltration.

When the sialylated HMO of interest is isolated in spray-dried form, it is preferably conducted as disclosed in e.g. WO 2013/185780, or the spray-drying process is performed e.g. with the 15-65 w/v % aqueous solution of the sialylated HMO of interest at a nozzle temperature of 110-190° C. and at an outlet temperature of 60-110° C., provided that the nozzle temperature is at least 10° C. higher than the outlet temperature.

### 3. Preferred Embodiments of the Invention

One embodiment of the method relates to obtaining or isolating a sialylated HMO, preferably 3′-SL or 6′-SL, from the fermentation broth in which it has been produced, comprising the steps of:

a) setting the pH of said reaction broth to pH=3-5,

b) optionally cooling the pH-adjusted broth obtained in step a) to 5-15° C. and stored for 1-15 days,

c) heating the broth of step a) or step b) to 60-65° C.,

d) contacting the broth obtained in step c) to an ultrafiltration (UF) membrane with a molecular weight cut-off (MWCO) of around 10-1000 kDa, wherein the membrane is a ceramic membrane, optionally at 40-65° C.,

e) optional sterile filtration of the permeate obtained in step d),

f) contacting the permeate obtained in step d) or the sterile filtered permeate obtained in step e) with a nanofiltration (NF) membrane having a MWCO of 600-3500 Da, wherein the active (top) layer of the membrane is composed of polyamide, and wherein the MgSO₄ rejection factor on said membrane is around 20-90%, preferably 50-90%,

g) treating the retentate obtained in step f) with a strong cationic ion exchange resin in H⁺-form followed by a weak basic ion exchange resin in base form to demineralize the retentate,

h) active charcoal chromatography of the solution obtained in step g) at 30-60° C., wherein the amount of the charcoal is around 2-10 weight % of the sialylated HMO contained in the load,

i) nanofiltration of the solution obtained in step h) with a membrane of 150-500 Da to concentrate the solution,

j) spray-drying the solution of step i) to obtain the sialylated HMO as an amorphous solid.

One embodiment of the method relates to obtaining or isolating a sialylated HMO, preferably 3′-SL or 6′-SL, from the fermentation broth in which it has been produced, comprising the steps of:

a) setting the pH of said reaction broth to a pH of around 5.5,

b) centrifuging the broth obtained in step a),

c) optionally heating the supernatant of step b) to 60-65° C.,

d) contacting the supernatant of step b) or step c) to an ultrafiltration (UF) membrane with a molecular weight cut-off (MWCO) of around 5-1000 kDa, optionally at 40-65° C.,

e) contacting the permeate obtained in step d) with a nanofiltration (NF) membrane
having a MWCO of 600-3500 Da, wherein the active (top) layer of the membrane is composed of polyamide, and wherein the MgSO₄ rejection factor on said membrane is around 20-90%, preferably 50-90%, or
having a MWCO of 150-500 Da,

f) active charcoal chromatography of the retentate obtained in step e) at 30-60° C., wherein the amount of the charcoal is around 2-10 weight % of the sialylated HMO contained in the load,

g) treating the solution obtained in step f) with a strong cationic ion exchange resin in H⁺-form followed by a weak basic ion exchange resin in base form to demineralize the solution,

h) nanofiltration of the solution obtained in step g) with a membrane of 150-500 Da to concentrate the solution,

i) spray-drying the solution of step i) to obtain the sialylated HMO as an amorphous solid.

In one preferred embodiment, the method relates to obtaining/isolating/purifying a sialylated HMO, preferably 3′-SL or 6′-SL, from the fermentation broth in which it has been produced, the method comprises the following sequence of steps consisting of steps A), B), C) and D):
A)—setting the pH of the broth to pH of about 3 to 5,
B)—centrifuging (CF) the pH-adjusted broth of step A) to give a clarified supernatant, or
   ultrafiltration (UF) of the pH-adjusted broth of step A) to give an ultrafiltration permeate (UFP),
C)—the clarified supernatant or UFP is treated with granulated active carbon to give a charcoal eluate (CCE), and
D)—nanofiltration (NF) of the CCE and collecting the nanofiltration retentate (NFR),

wherein the method does not comprise ion exchange treatment, simulated moving bed chromatography, gel filtration/chromatography and electrodialysis.

This method comprises less purification steps than previous prior art methods and surprisingly provides a sialylated HMO, preferably 3′-SL or 6′-SL, that meets strict regulatory specifications.

Especially, the method does not comprise ion exchange treatment that is usually the part of the purification of such substances. Therefore, a sialylated HMO, preferably 3′-SL or 6′-SL, can be provided for human consumption in a more economic way.

In the method specified above, the acid that can be used for pH-adjustment in step A) is sulfuric acid. For example, the acid could be 20% H₂SO₄-solution. However, other acids can be used as well, and the particular acid used is not limiting. For example, the acid can be selected from one or more of H₂SO₄, HCl, H₃PO₄, formic acid, acetic acid and citric acid. Each of these acids can be used alone or in combination with any other acids.

In step B), centrifugation or UF is equally applicable. These methods are taught in detail above.

In step C), the amount of granulated active carbon is selected so that the sialylated HMO, preferably 3′-SL or 6′-SL, is not, or at least not substantially, adsorbed on the charcoal particles and elution with water gives rise to an aqueous solution of the sialylated HMO, preferably 3′-SL or 6′-SL, without its significant loss, meanwhile the colour giving substances remain adsorbed. To achieve this, the amount of activated charcoal applied for decolourization should be about 12-25% by mass relative to the sialylated HMO, preferably 3′-SL or 6′-SL, content of the feed solution obtained in the previous step, preferably about 15-20% relative to the sialylated HMO, preferably 3′-SL or 6′-SL, content of the feed solution. With this particular arrangement, as much as at least 90% of the sialylated HMO, preferably 3′-SL or 6′-SL, (by mass) obtained in the previous step can be collected back in the form of a decolourized solution. Moreover, under the conditions disclosed above, a significant reduction of the protein content of the feed solution is achievable, such as at least the 90% of the residual protein is removable, preferably around 98-99% of the residual protein can be removed. Therefore, a low protein charcoal eluate is provided to the next NF step.

In step D), the NF step is preferably conducted according to the preferred and more preferred embodiments of second aspect disclosed above in detail. In this step, lactose, sialic acid and other similar low molecular weight organic compounds can be separated from the sialylated HMO, preferably 3′-SL or 6′-SL, and significant amount of mono- and divalent ions, preferably inorganic ions, is also removable. An example of a suitable NF membrane is the piperazine based polyamide TFC membrane TriSep® UA60.

According to the above procedure, the sialylated HMO, preferably 3′-SL or 6′-SL, in the NF retentate is obtainable in the purity that meets the regulatory specifications.

### EXAMPLES

### Example 1

6′-SL was made by fermentation using a genetically modified E. coli cell of LacZ⁻, LacY⁺ phenotype carrying heterologous neuBCA, wherein said cell comprises a recombinant gene encoding an α-2,6-sialyl transferase which is able to transfer the sialic acid of GMP-sialic acid to the internalized lactose, and deleted or inactivated nanKETA. The fermentation was performed by culturing said cell in the presence of exogenously added lactose and a suitable carbon source, thereby producing 6′-SL. The obtained fermentation broth containing 6′-SL (pH is around 6.3) was acidified with sulfuric acid to around 4.5. The acidified broth was divided into two fractions, each of which was separately centrifuged at 8000 RCF for 1 h at 20° C. The table below shows the amount of supernatants obtained from the fractions and the corresponding properties.

| | | | | | Protein |
|---|---|---|---|---|---|
| | | Amount of | Brix of | | content |
| Fraction | BWM of | supernatant | supernatant | | Bradford |
| # | broth | (kg) | (°Bx) | pH | (mg/l) |
| 1 | 31.5% | 1.85 | 6.3 | 4.48 | 1172.7 |
| 2 | 44.3% | 1.54 | 12.8 | 4.65 | 834.1 |

The supernatants of fraction #1 and fraction #2 were mixed and diluted using distilled water to generate a protein concentration of ca. 300 mg/l. This diluted supernatant mixture was used as a feed to process on a packed bed granulated activated carbon (GAC, i.e. CPGLF 12×40, 175 g). Before the start of the GAC operation the packed bed GAC was back flushed (i.e. in up-flow mode) using hot distilled water at 90° C. for 2 h. Table 2 summarizes the GAC feed properties and process conditions. Table 3 summarizes results in terms of bulk and solute material balance as well as GAC effluent properties. Moreover, the entire GAC effluent was collected immediately at the beginning of processing (i.e. including the void volume) while at the end of operation the liquid in the GAC bed was pushed out by simply pumping air through the bed. Therefore, as can be seen in Table 3, it is expected that the GAC effluent is diluted (i.e. the bulk material balance is larger than 100%).

| | | | Protein | | | | | | |
|---|---|---|---|---|---|---|---|---|---|
| | | | content, | | CI | | | | |
| | Amount | [6′-SL] | Bradford | Brix | @400 | | 6′-SL | [lactose] | [sialic acid] |
| | (kg) | (wt. %) | (mg/l) | (°Bx) | nm | pH | yield | (wt. %) | (wt. %) |
| feed | 9.84 | 1.36 | 276.6 | 2.9 | 528.5 | 4.5 | — | | |
| eluate | 11.41 | 1.03 | 2.9 | 2.5 | 15.8 | 4.9 | 88% | 0.26 | 0.044 |

The eluate was concentrated in a rotavapor at 65° C. and ca. 50 mbar and processed to NF/DF. The NF/DF process was performed in a batch mode using a spiral-wound thin-film composite (TFC) membrane TRISEP TurboClean 1812-UA60 with area of 0.23 m². Operation parameters were TMP ca. 39 bar and cross flow of 400 l/h while the temperature was dynamic but was maintained to be below 45° C. The table below summarizes the properties of the final homogenized NF/DF retentate.

| NF feed | DF water | 6′-SL | Brix | | conductivity | lactose | sialic acid |
|---|---|---|---|---|---|---|---|
| (kg) | (kg) | yield | (°Bx) | pH | (mS/cm) | removal | removal |
| 1.73 | 9.7 | 82% | 11.7 | 4.12 | 8.01 | 93% | 79.5% |

The NF/DF retentate was freeze-dried having the final product quality:

| | 6′-SL | lactose | sialic acid | protein | water | Cl⁻ | sulphate |
|---|---|---|---|---|---|---|---|
| | (%) | (%) | (%) | (ppm) | (%) | (%) | (%) |
| regulatory | ≥90 | ≤5 | ≤2 | ≤10000 | ≤6 | ≤1.0 | ≤1.0 |
| requirement | | | | | | | |
| example | 93.8 | 1.95 | 1.14 | 25 | 2 | 0.05 | 1 |

The data demonstrate that the purification of 6′-SL from fermentation broth using pH-adjustment, centrifugation, active charcoal decolorization and nanofiltration provides a product that meets the regulatory specifications, especially the protein content was reduced significantly.

The concentration/amount of the carbohydrate compounds were determined by HPLC ion chromatography: TSKGel Amide-80 (150 mm×4.6 mm, 3 μm) with 70% acetonitrile, 15% water and 15% 10 mM ammonium formate buffer at a flow rate of 1.1 ml/min and 25° C. using charged aerosol detection (CAD).

### Example 2—Determination of a Substance Rejection Factor on a Membrane

The NaCl and MgSO₄ rejection on a membrane is determined as follows: in a membrane filtration system, a NaCl (0.1%) or a MgSO₄ (0.2%) solution is circulated across the selected membrane sheet with (for Tami: tubular module) while the permeate stream is circulated back into the feed tank. The system is equilibrated at 10 bars and 25° C. for 10 minutes before taking samples from the permeate and retentate. The rejection factor is calculated from the measured conductivity of the samples: (1−κₚ/κᵣ) 100, wherein κₚ is the conductivity of NaCl or MgSO₄ in the permeate and κᵣ is the conductivity of NaCl or MgSO₄ in the retentate.

| | | | NaCl rej. factor | | MgSO₄ rej. factor | |
|---|---|---|---|---|---|---|
| | | | supplier | lab. | supplier | lab. |
| membrane | active layer | MWCO | spec. | measurement | spec. | measurement |
| Trisep UA60 | piperazine-PA | 1000-3500 | — | 10% | 80% | 81-89% |
| GE GH | PA | 2500 | — | 81% | — | 76% |
| NTR-7450 | sulph. PES | 600-800 | 50% | 56% | — | 32% |
| Tami | ceramic | 1000 | — | — | — | 0% |

The rejection factor of a carbohydrate, e.g. a sialylated HMO such as 3′-SL or 6′-SL, is determined in a similar way with the difference that the rejection factor is calculated from the concentration of the samples (determined by HPLC): (1−Cₚ/Cᵣ) 100, wherein Cₚ is the concentration of the carbohydrate in the permeate and Cᵣ is the concentration of the carbohydrate in the retentate.

1. A method for purifying a sialylated human milk oligosaccharide (HMO) from the reaction milieu in which it has been produced, comprising the steps of:
pre-treating the reaction milieu via pH-adjustment, dilution and/or heat treatment, and
centrifuging the reaction milieu after the pre-treatment or contacting the reaction milieu after the pre-treatment to an ultrafiltration (UF) membrane with a molecular weight cut off (MWCO) of around 5-1000 kDa.

2. The method of claim 1, wherein the pre-treatment comprises adjusting the pH of the reaction milieu to 3-5.

3. The method of claim 1, further comprising the step of contacting
the clarified supernatant obtained from centrifugation or
the UF permeate with a nanofiltration (NF) membrane.

4. The method of claim 3, further comprising the step of a strong cation exchange resin treatment in H+-form and a weak anion exchange resin treatment in free base form.

5. The method of claim 2, wherein the reaction milieu is a fermentation broth in which the sialylated HMO has been produced and wherein the clarified supernatant obtained from centrifugation or the UF permeate is treated with granulated active carbon to give a charcoal eluate and the charcoal eluate is nanofiltered to collect the nanofiltration retentate, and wherein the method does not comprise ion exchange treatment, simulated moving bed chromatography, gel filtration/chromatography and electrodialysis.

6. The method of claim 3, wherein the NF membrane has a molecular weight cut-off (MWCO) of 600-3500 Da, the active (top) layer of the NF membrane is composed of polyamide, and wherein the MgSO₄ rejection factor on said membrane is around 50-90%.

7. The method of claim 1, comprising
a) optionally, centrifugation, microfiltration of the reaction milieu, or filtration of the reaction milieu on a filter press or a drum filter,
b) as pre-treatment, setting the pH of the filtrate or supernatant from step a) or the reaction milieu directly to 3-6 and/or warming up the filtrate or supernatant from step a) or the reaction milieu directly to 35-65° C., and
c) contacting the reaction milieu obtained in step b) to an ultrafiltration (UF) membrane with a molecular weight cut-off (MWCO) of 5-1000 kDa, such as 10-1000 kDa and collecting the permeate,
with the proviso that when step a) is not carried out, then the UF membrane is a non-polymeric membrane.

8. The method of claim 7, wherein step a) is not carried out and wherein the non-polymeric membrane is a ceramic membrane.

9. The method of claim 7, wherein step c) is carried out at 45-65° C.

10. The method of claim 9, wherein the UF membrane is a ceramic membrane, and the method further comprises the step of contacting the permeate obtained in step c) with a nanofiltration (NF) membrane and collecting the retentate.

11. The method of claim 10, wherein the NF membrane has a molecular weight cut-off (MWCO) of 600-3500 Da, the active top layer of the NF membrane is composed of polyamide, and wherein the MgSO₄ rejection factor on said membrane is around 50-90%.

12. The method of claim 10, wherein the nanofiltration retentate is treated with an ion exchange resin, the treatment with ion exchange resin consists of the treatment of the NF retentate with a strong cation exchange resin in H+-form directly followed by a treatment with a weak anion exchange resin in free base form.

13. The method of claim 10, wherein the nanofiltration retentate is treated with active charcoal, wherein the amount of the charcoal is around 2-10 weight % of the sialylated HMO contained in the nanofiltration retentate, to give an active charcoal eluate.

14. The method of claim 12, wherein the eluate of the ion exchange treatment is treated with active charcoal, wherein the amount of the charcoal is around 2-10 weight % of the sialylated HMO contained in the eluate, to give an active charcoal eluate.

15. The method of claim 13, wherein the active charcoal eluate is treated with an ion exchange resin, the treatment with ion exchange resin consists of the treatment of the active charcoal eluate with a strong cation exchange resin in H⁺-form directly followed by a treatment with a weak anion exchange resin in free base form.

16. The method of claim 1, wherein the reaction milieu is a fermentation broth obtained by culturing a genetically modified cell capable of producing said sialylated HMO from an internalized carbohydrate precursor.

17. The method of claim 16, wherein the genetically modified microorganism is an E. coli of LacZ⁻ genotype.

18. The method of claim 17, wherein the E. coli comprises a recombinant α-2,3- or α-2,6-sialyl transferase.

19. The method of claim 18, wherein the E. coli carries neuBCA genes.

20. The method of claim 1, wherein the sialylated HMO is 3′-SL or 6′-SL.
